(19) United States
(12) Patent Application Publication
(10) Pub. No.: US 2009/0005267 A1
(43) Pub. Date: Jan. 1, 2009

(54) IMMUNOASSAY FOR CROSS-REACTING SUBSTANCES

(75) Inventors: Bradley Love, Timonium, MD (US); Brian Dwyer, San Diego, CA (US); George Klee, SW Rochester, MN (US); Joseph Beechem, Eugene, OR (US); Ravinder Singh, Rochester, MN (US); Stefan Grebe, Rochester, MN (US)

Correspondence Address: INVITROGEN CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 12/041,482

(22) Filed: Mar. 3, 2008

Related U.S. Application Data
(60) Provisional application No. 60/892,323, filed on Mar. 1, 2007.

(51) Int. Cl. C40B 40/10; G01N 33/53; G01N 33/536; G01N 33/543; G06F 19/00; G01N 33/545; G01N 33/544

(52) U.S. Cl. 506/18; 436/536; 435/7.1; 435/7.9; 436/518; 436/531; 436/528; 702/19

(57) ABSTRACT

The present disclosure provides an immunoassay involving a multiplex of antibodies that recognize the same analyte but that have a different cross-reactivity to structurally similar compounds. Data obtained from the immunoassay involving observed analyte concentrations is input into an algorithm to determine the true concentration of the analyte in a sample.

### FIELD OF THE INVENTION

Immunoassays for the detection and quantification of an analyte in a solution comprising cross-reactive ligands are disclosed. In one particular embodiment, the analyte is a steroid, such as cortisol and the cross-reactive ligands are non-cortisol steroids.

### BACKGROUND OF THE INVENTION

Cortisol is a potent glucocorticoid produced by the human adrenal gland. It is synthesized from cholesterol and its production is stimulated by pituitary adrenocorticotropic hormone (ACTH) which is regulated by corticotropin releasing factor (CRF). Cortisol acts through specific intracellular receptors and affects numerous physiologic systems including immune function, glucose counter regulation, vascular tone, and bone metabolism.

Elevated cortisol levels and lack of diurnal variation have been identified with Cushing's disease (ACTH hypersecretion). Elevated circulating cortisol levels have also been identified in patients with adrenal tumors. Low cortisol levels are found in primary adrenal insufficiency (e.g. adrenal hypoplasia, Addison's disease) and in ACTH deficiency. Cortisol or hydrocortisone, along with several other analogs such as Prednisone, are also administered parenterally for treatment of a variety of disorders. Accordingly, monitoring of cortisol levels is critical in a number of clinical situations.

Cortisol belongs to a class of corticosteroids that are structurally very similar. Accordingly, immunoassays for cortisol are subject to interference from cross-reacting substances. Particularly, prednisolone is so chemically similar to cortisol that many existing analytical methods cannot distinguish between the two steroids (Thorax 2000; 55, 722). Similarly, assays for other non-cortisol substrates, such as prednisolone, dexamethasone, herbicidal triazines (J. Agric. Food Chem. 1990, 38, 433-437), and human T-cell lymphotropic virus (HTLV) (Clinical and Diagnostic Laboratory Immunology, 1998, 5(1), 45-49), suffer from interference with other structurally similar compounds. The result of cross-reactivity in immunoassays can result in severe miscalculations of substrate concentrations that can lead to incorrect clinical decisions.

Accordingly, a need exists for determining the true concentration of an analyte in an immunoassay prone to interference with cross-reactive substances. In particular a need exist for the detection of true cortisol levels in a biological sample containing cortisol and non-cortisol steroids.

### SUMMARY OF THE INVENTION

Immunoassays for cortisol are subject to interference from cross-reacting substances such as structurally similar glucocorticoids and synthetic steroids. This interference can result in erroneously high results with negative consequences. The present invention provides multiplexed assays for cortisol where anti-cortisol and other anti-steroid antibodies with different cross-reactivity profiles are present in the multiplex. The assay response for each antibody is assessed, and the apparent cortisol concentrations obtained from each assay are input together into an algorithm designed to extract the true cortisol concentration. The algorithm is developed by analyzing synthetic mixtures of cortisol and the relevant cross-reacting steroids. The assay is designed to be quantitative for the purpose of assaying patient samples in matrices including plasma, serum, saliva and urine.

One embodiment of the present invention provides a method for determining the concentration of an analyte in a test sample comprising the analyte and a plurality of competitive ligands, the method comprising:

contacting the test sample with at least two different anti-analyte antibodies, wherein each of the antibodies bind the analyte and have a different level of cross-reactivity for the competitive ligands;

detecting binding of the analytes and competitive ligands to the antibodies, thereby determining an observed analyte binding amount for each antibody; and

performing a regression analysis on the observed analyte binding amount for each antibody to determine the concentration of the analyte in the test sample.

Another embodiment of the present invention provides a method for determining the concentration of cortisol in a test sample, the method comprising:

contacting the test sample with at least two different anti-steroid antibodies, wherein each of the antibodies bind cortisol and have a different level of cross-reactivity with non-cortisol steroids;

detecting binding of steroids to the antibodies, thereby determining an observed steroid binding amount for each antibody;

performing a regression analysis on the observed steroid binding amounts for each antibody to determine the concentration of cortisol in the test sample.

Another embodiment of the present invention provides a composition comprising at least five different isolated anti-steroid antibodies, wherein each of the antibodies bind cortisol and have a different level of cross-reactivity with non-cortisol steroids.

Another embodiment of the present invention provides an array device comprising a solid support comprising at least two different anti-steroid antibodies, wherein each of the antibodies bind cortisol and have a different level of cross-reactivity with non-cortisol steroids.

Another embodiment of the present invention provides a kit for determining cortisol concentration in a test sample comprising:

a solid support comprising at least two different anti-steroid antibodies, wherein each of the antibodies bind cortisol and have a different level of cross-reactivity with non-cortisol steroids; and

instructions on how to determine the cortisol concentration in the test sample.

Another embodiment of the present invention provides a method for detecting cortisol levels in an individual, the method comprising:

contacting a test sample from the individual with at least two different anti-steroid antibodies, wherein each of the antibodies bind cortisol and have a different level of cross-reactivity with non-cortisol steroids;

detecting binding of steroids to each of the antibodies, thereby determining an observed steroid binding amount for each of the antibodies;

performing a regression analysis on the observed steroid binding amounts for each antibody to determine the concentration of cortisol in the test sample; and

comparing the concentration of cortisol in the test sample from the individual with cortisol levels in a control sample to detect cotisol levels in the individual.

Another embodiment of the present invention provides a method of using a computer processor to determine the concentration of cortisol in a test sample, the method comprising:

receiving data representing observed steroid concentrations in a test sample, wherein the data is obtained from contacting at least two different anti-steroid antibodies with a test sample, wherein each of the antibodies bind cortisol and have a different level of cross-reactivity with non-cortisol steroids; and

performing a linear regression analysis with the computer processor with the data to determine a result comprising the concentration of cortisol in the test sample.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 depicts cross reactivity on planar microarray by spiking study.

FIG. 2 depicts the predicted vs the true concentration in a sample.

FIG. 3 (FIG. 3A=Ab 7; FIG. 3B=Ab 9; FIG. 3C=Ab 10; FIG. 3D=Ab 39; FIG. 3E=Ab 40; and FIG. 3F=Ab 41) depict the true cortisol amount versus the single antibody estimated concentration (denoted with an x for each sample), the antibody prediction line (which does not intersect the Y-axis at 0) is the overall trend between estimated and actual concentration of cortisol for all samples and the perfect prediction line (which does intersect the Y-axis at 0) is the line that represents a trend of perfect prediction of cortisol. The circles show the estimated cortisol concentration versus actual concentration with the reduced regression model.

FIG. 4 encompasses the cross reacting species concentration showing the prediction of prednisone and prednisolone in a given sample.

### DETAILED DESCRIPTION OF THE INVENTION

### Introduction:

Shortcomings of immunoassays are usually reflective of their poor specificity and selectivity. These attributes are due usually to the immunological limitations which antibodies have in immunodiagnostic systems. In the case of steroidal compounds, cortisol is a commonly used analyte to diagnose Cushing's syndrome and other hormonal diseases. There are several commonly prescribed medications administered to patients which may interact with the current commercially available immunoassay systems and cause erroneous results.

Accordingly, mass spectrometric methods have been developed to resolve and identify the cross reacting nature induced from endogenous and exogenous species. By using mass spectrometry coupled with chromatographic systems the cross reactivity is completely side-stepped. However, mass spectrometry is generally considered to expensive and time consuming. It is also limited to a number of locations for clinical testing.

In the present method mass spectrometric methods were used to determine actual analyte levels in clinical patient populations. Some of these samples were subjected to the current immunoassay systems and the interference relationship was established. After identifying the most commonly present exogenous drugs present in the samples, this information was used toward establishing an assay, free from the limitations of the mass spec. deconvolution, that gives an accurate depiction of the analyte, such as cortisol, levels in a patient sample.

Various antibodies were selected to either enhance or suppress the affinity for various interfering compounds. This gave cross-reactivity levels which were used to back calculate the true analyte concentration from the sample signal profile exhibited. This was achieved after characterization of the antibodies for the various compounds was concluded. Once the outline was laid then a regression analysis was performed to calculate the true cortisol concentration in the sample.

### Definitions

Before describing the present invention in detail, it is to be understood that this invention is not limited to specific compositions or process steps, as such may vary. It should be noted that, as used in this specification and the appended claims, the singular form “a”, “an” and “the” include plural references unless the context clearly dictates otherwise. Thus, for example, reference to “a steroid” may include a plurality of steroids and reference to “an antibody” may include a plurality of antibodies and the like.

Unless defined otherwise, all technical and scientific terms used herein have the same meaning as commonly understood by one of ordinary skill in the art to which this invention is related. The following terms are defined for purposes of the invention as described herein.

“Analyte” refers to a material, such as cortisol, for which the assay aims to detect or quantify.

“Antibody 7” refers to clone XM210 from Abcam. “Antibody 9” refers to clone F4P1A3 from EMD Biosciences. “Antibody 10” refers to clone A29220314P from BiosPacific. Antibody 39 and 40 are polyconal antibodies for Cortisol 21-HS BSA and

Antibody 41 and 42 are prednisolone 21-HS BSA conjugates from immunized rabbits (each from different rabbits).

“Cross-reactive” or “cross-reactivity” as used herein refers to the binding of multiple different ligands with a single antibody or receptor. In the present invention, antibody's cross-reactivity level can be predetermined. The less discriminate an antibody is for a particular analyte, as compared with competitive ligands (i.e. analogs of the analyte), the greater the cross-reactivity.

“Test sample” as used herein refers to media, such as blood or a control, that may have an analyte of interest, such as cortisol.

“Competitive ligands” refer to at least one material that competes with an analyte of interest for a particular target (i.e. is cross-reactive). One example of a competitive ligand for cortisol is prednisolone.

A “regression analysis” involves modeling relationships between variables, such as observed binding amounts for antibodies, to determine the relationship between the variables. The regression analysis can be linear or non-linear. Further description of regression analyses are provided herein.

“Stereoisomer” or “stereoisomers” refer to compounds that differ in the chirality of one or more stereocenters. Stereoisomers include enantiomers and diastereomers.

The term “isolated” means that the material is removed from its original environment (e.g., the natural environment if it is naturally occurring). For example, a naturally-occurring steroid present in a living animal is not isolated, but the same steroid, separated from some or all of the coexisting materials in the natural system, is isolated. Such steroids could be part of a composition, and still be isolated since the composition is not part of its natural environment.

“Tautomer” refers to alternate forms of a compound that differ in the position of a proton, such as enol-keto and imine-enamine tautomers, or the tautomeric forms of heteroaryl groups containing a ring atom attached to both a ring —NH— moiety and a ring ═N— moeity such as pyrazoles, imidazoles, benzimidazoles, triazoles, and tetrazoles.

“Patient,” “subject” or “individual” refers to mammals and includes humans and non-human mammals, such as monkeys, dogs, cats, horses, cows, pigs or rats.

“Salt” refers to salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate.

“Treating” or “treatment” of a disease in a patient refers to 1) preventing the disease from occurring in a patient that is predisposed or does not yet display symptoms of the disease; 2) inhibiting the disease or arresting its development; or 3) ameliorating or causing regression of the disease.

The terms “protein” and “polypeptide” are used herein in a generic sense to include polymers of amino acid residues of any length. The term “peptide” is used herein to refer to polypeptides having less than 250 amino acid residues, typically less than 100 amino acid residues. The terms apply to amino acid polymers in which one or more amino acid residues are an artificial chemical analogue of a corresponding naturally occurring amino acid, as well as to naturally occurring amino acid polymers.

The term “reactive group” as used herein refers to a group that is capable of reacting with another chemical group to form a covalent bond, i.e. is covalently reactive under suitable reaction conditions, and generally represents a point of attachment for another substance. The reactive group is a moiety, such as carboxylic acid or succinimidyl ester, on the compounds of the present invention that is capable of chemically reacting with a functional group on a different compound to form a covalent linkage. Reactive groups generally include nucleophiles, electrophiles and photoactivatable groups.

Exemplary reactive groups include, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like. Methods to prepare each of these functional groups are well known in the art and their application to or modification for a particular purpose is within the ability of one of skill in the art (see, for example, Sandler and Karo, eds., Organic Functional Group Preparations, Academic Press, San Diego, 1989).

The term “detectable response” as used herein refers to an occurrence of or a change in, a signal that is directly or indirectly detectable either by observation or by instrumentation. Typically, the detectable response is an optical response resulting in a change in the wavelength distribution patterns or intensity of absorbance or fluorescence or a change in light scatter, fluorescence lifetime, fluorescence polarization, or a combination of the above parameters.

The term “dye” as used herein refers to a compound that emits light to produce an observable detectable signal.

The term “fluorophore” or “fluorescent label” as used herein refers to a composition that is inherently fluorescent or demonstrates a change in fluorescence upon binding to a biological compound or metal ion, or metabolism by an enzyme. Preferred fluorophores of the present invention include fluorescent dyes having a high quantum yield in aqueous media. Exemplary fluorophores include xanthene, indole, borapolyazaindacene, furan, and benzofuran, among others. The fluorophores of the present invention may be substituted to alter the solubility, spectral properties or physical properties of the fluorophore.

Labels that can be used herein for detection are known by those of skill in the art and include, but are not limited to, radiolabels, pigments, dyes or other chromogens, spin labels, fluorescent compounds, haptens, electron transfer agents, and particles. The label can also be a precursor to a luminescent substance; a bioluminescent substance; a chemiluminescent substance, or a metal-containing substance. Preferred labels are fluorescent moieties including xanthenes, cyanines, coumarins, indoliniums, coumarins, benzofurans, borapolyazaindacene, as well as those described in the MOLECULAR PROBES HANDBOOK OF FLUORESCENT PROBES AND RESEARCH CHEMICALS by R. P. Haugland 10^{th} Ed., (2005).

Preferred enzyme substrates of the invention are enzyme substrates that yield a fluorescent product that localizes at or near the site of enzyme activity. Enzymes of use in the method include any enzymes that utilize a chromogenic (e.g. DAB or FastRed with HRP or AP), fluorogenic or chemiluminescence-generating substrate. Preferred enzymes of the invention include peroxidases, phosphatases, glycosidases, aequorins, or luciferases, and more specifically, HRP, Coprinus cinereus peroxidase, Arthromyces ramosus peroxidase, alkaline phosphatase, β-galactosidase, β-glucuronidase, or a protein A or protein G fusion protein of luciferase.

Illumination of the test sample at a suitable wavelength results in one or more illuminated targets that are then analyzed according to the response of their fluorescence to the illumination. The illuminated targets are observed with any of a number of means for detecting a fluorescent response emitted from the illuminated target, including but not limited to visual inspection, cameras and film or other imaging equipment, or use of instrumentation such as fluorometers, plate readers, laser-based scanners, microscopes, or flow cytometers, or by means for amplifying the signal such as a photomultiplier (PMT).

The analyte of interest, a fluorescent labeled version, or other derivatives, analogs thereof, or competitive ligands are used as an immunogens to produce antibodies described herein. These antibodies are, for example, polyclonal or monoclonal antibodies. The present invention also includes chimeric, single chain, and humanized antibodies, as well as Fab fragments, or the product of a Fab expression library. Various procedures known in the art may be used for the production of such antibodies and fragments.

Antibodies generated against the immunogens, can be obtained by direct injection of the immunogen into an animal or by administering the immunogen to an animal, preferably a nonhuman. The antibody so obtained will then bind the immunogen itself as well as competitive ligands with varying affinity (for each antibody). In this manner, a degree or level of cross-reactivity can be determined for an individual or set of antibodies.

For preparation of monoclonal or polyclonal antibodies, any technique which provides antibodies produced by continuous or multiple cell line cultures can be used. Examples include the hybridoma technique (Kohler and Milstein, 1975, Nature, 256:495-497), the trioma technique, the human B-cell hybridoma technique (Kozbor et al., 1983, Immunology Today 4:72), and the EBV-hybridoma technique to produce human monoclonal antibodies (Cole, et al., 1985, in Monoclonal Antibodies and Cancer Therapy, Alan R. Liss, Inc., pp. 77-96).

For example, monoclonal antibodies may be generated by immunizing an animal (e.g., mouse, rabbit, etc.) with a desired antigen/analyte and the spleen cells from the immunized animal are immortalized, commonly by fusion with a myeloma cell. Immunization with antigen may be accomplished in the presence or absence of an adjuvant, e.g., Freund's adjuvant. Typically, for a mouse, 10 μg antigen in 50-200 μl adjuvant or aqueous solution is administered per mouse by subcutaneous, intraperitoneal or intra-muscular routes. Booster immunization may be given at intervals, e.g., 2-8 weeks. The final boost is given approximately 2-4 days prior to fusion and is generally given in aqueous form rather than in adjuvant.

Spleen cells from the immunized animals may be prepared by teasing the spleen through a sterile sieve into culture medium at room temperature, or by gently releasing the spleen cells into medium by pressure between the frosted ends of two sterile glass microscope slides. The cells are harvested by centrifugation (400×g for 5 min.), washed and counted. Spleen cells are fused with myeloma cells to generate hybridoma cell lines. Several mouse myeloma cell lines which have been selected for sensitivity to hypoxanthine-aminopterin-thymidine (HAT) are commercially available and may be grown in, for example, Dulbecco's modified Eagle's medium (DMEM) (Gibco BRL) containing 10-15% fetal calf serum. Fusion of myeloma cells and spleen cells may be accomplished using polyethylene glycol (PEG) or by electrofusion using protocols which are routine in the art. Fused cells are distributed into 96-well plates followed by selection of fused cells by culture for 1-2 weeks in 0.1 ml DMEM containing 10-15% fetal calf serum and HAT. The supernatants are screened for anti-analyte (e.g. cortisol) antibody production using methods well known in the art. Hybridoma clones from wells containing cells which produce antibody are obtained, e.g., by limiting dilution. Cloned hybridoma cells (4-5×10⁶) are implanted intraperitoneally in recipient mice, preferably of a BALB/c genetic background. Sera and ascites fluids are collected from mice after 10-14 days.

Polyclonal antibodies are produced by immunizing a mouse, rabbit, chicken, or other animal. The antigen/analyte is injected into the animal along with a suitable adjuvant, such as Freund's adjuvant. Immunization results in the production of antibodies specific to that antigen. The animal serum may be used as the product or the antibodies may be purified from the serum. The polyconal antibodies can be produced with an average cross-reactivity over the group. Accordingly, a batch of antibodies with minor variances in cross-reactivity can still have a single cross-reactivity level across the group.

The term “mutation” or “mutant” as used herein refers to a change in the genotype that leads to a different protein, in particular, from a different antibody coding sequence. The mutation may be a deletion, insertion, point mutation, or any other detectable change in the wild-type form of the protein.

The invention also contemplates humanized antibodies which may be generated using methods known in the art, such as those described in U.S. Pat. Nos. 5,545,806; 5,569,825 and 5,625,126, the entire contents which are incorporated by reference. Such methods include, for example, generation of transgenic non-human animals which contain human immunoglobulin chain genes and which are capable of expressing these genes to produce a repertoire of antibodies of various isotypes encoded by the human immunoglobulin genes.

Techniques described for the production of single chain antibodies (U.S. Pat. No. 4,964,778) can be adapted to produce single chain antibodies to immunogenic polypeptide products of this invention. Also, transgenic mice may be used to express humanized antibodies to immunogenic polypeptide products of this invention.

The term “fragment,” when referring to the antibodies of the present invention, means antibody fragments which retain essentially the same biological function or activity as the full size antibody. Thus, in one embodiment a fragment of an antibody includes just the Fab or light chain portion of the antibody that is capable of binding to the analyte of interest (e.g. cortisol).

A fragment or “derivative” or “analog” may be a polypeptide in which one or more of the amino acid residues are substituted with a conserved or non-conserved amino acid residue (preferably a conserved amino acid residue) and such substituted amino acid residue may or may not be one encoded by the genetic code, or one in which one or more of the amino acid residues includes a substituent group, or one in which the mature polypeptide is fused with another compound, such as a compound to increase the half-life of the polypeptide (for example, polyethylene glycol), or one in which the additional amino acids are fused to the mature polypeptide, such as a leader or secretory sequence or a sequence which is employed for purification of the mature polypeptide or a protein sequence. Such fragments, derivatives and analogs are deemed to be within the scope of those skilled in the art from the teachings herein.

The polypeptides and antibodies of the present invention are preferably provided in an isolated form, and preferably are purified to homogeneity.

Polynucleotides may be employed for producing polypeptides by recombinant techniques. Thus, for example, the polynucleotide may be included in any one of a variety of expression vectors for expressing a polypeptide. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host.

The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

The antibodies and fragments thereof described herein may be utilized for in vitro purposes related to scientific research and for designing therapeutics, such as cortisol analogues, for the treatment of human disease.

Aspects of the present invention relate particularly to an assay for detecting true levels of cortisol in test samples comprising close structural analogues to cortisol, such as prednisolone. The present assay takes advantage of the fact that antibodies can have measurable and predeterminable cross-reactivity values for the competitive ligands which can be compared in a regression analysis to calculate the true cortisol levels in a sample. Preferably the assay comprises a competitive-binding assays, however additional assays known to those of skill in the art such as immunohistochemical (IHC) analysis, radioimmunoassays, Western Blot analysis, ELISA assays and “sandwich” assays are contemplated as potential assay formats.

In one embodiment of the invention, bound analytes are visualized by immunohistochemistry by localizing analytes in cells of a tissue section for binding to their respective antibodies. Visualization is enabled by tagging the antibody with color producing labels. Some labels include Horseradish peroxidase or alklaline phosphatase. An ideal chemistry produces the required color using different redox dyes. Alternatively, the antibody can also be tagged to different fluorophores. The fluorophores can be used in conjunction confocal laser scanning microscopy for sensitive visualization of two interacting protein molecules together.

In another embodiment, an ELISA assay is used, which initially comprises preparing antibodies with varying cross-reactivity for a particular analyte. In addition a reporter antibody is prepared against the polyclonal or monoclonal antibody. To the reporter antibody is attached a detectable reagent such as fluorescence or, in this example, a horseradish peroxidase enzyme. A sample is removed from a host and incubated on a solid support, e.g. a polystyrene dish, that binds the analytes and competitive ligands in the sample. All unbound monoclonal or polyclonal antibody is washed out with buffer and preferably, unbound sites blocked. The reporter antibody linked to horseradish peroxidase is now placed in the dish resulting in binding of the reporter antibody to any monoclonal or polyclonal antibody bound to the analyte and or competitive ligands. Unattached reporter antibody is then washed out. Peroxidase substrates are then added to the dish and the amount of color developed in a given time period is a measurement of the observed analyte amount present in a given volume of test sample. A regression analysis is then performed as described herein and the true analyte amount is determined. Preferably the analyte is cortisol.

A competition assay is preferably performed as described in greater detail throughout the specification, wherein anti-analyte antibodies are optionally attached to a solid support and labeled analytes and/or label competitive ligands and a sample derived from the host are passed over the solid support and the amount of label detected, for example by liquid scintillation chromatography, can be correlated to an observed analyte amount in the sample.

A “sandwich” assay is similar to an ELISA assay. In a “sandwich” assay, analyte is passed over a solid support and binds to antibody attached to a solid support. A second antibody is then bound to the analyte. A third antibody which is labeled and specific to the second antibody is then passed over the solid support and binds to the second antibody and an amount can then be quantified.

The invention also provides methods which initially involve pre-forming the immunolabeling complex, the target-binding antibody and the labeling protein, followed by addition to a biological sample and determination of the desired target. The immunolabeling complex is pre-formed, which contains the target binding antibody and the labeling protein bound by a detectable label, followed by the addition to a sample suspected of containing the desired target. The labeling protein is a monovalent protein, either a Fab fragment or a non-immunoglobulin peptide or protein that specifically binds the Fc region of the target-binding antibody. The labeling protein is covalently attached to one or more detectable labels, wherein the detectable labels can be the same or different allowing for multiparameter applications. Addition of the pre-formed immunolabeling complex to a sample, followed by sufficient time for the complex to bind with the target, detection of the label is determined. Methods of visualizing the label depend on the label attached to the labeling protein.

Anti-steroid antibodies including antibodies against cortisol as well as cross-reacting steroids have different or complementary cross-reactivity profiles in order to provide the data necessary for the algorithm. The commercially available anti-cortisol antibodies are generated with the 3-carboxymethyloxime derivative of cortisol. While this yields antibody specificity that suffices for an ELISA assay utilizing one unique antibody, the multiplexed array requires a diverse collection of unique antibodies. This utilizes antibodies that are generated against a number of alternative cortisol conjugates, for example the 3-carboxymethyloxime and 21-hemisuccinate derivatives as well as similar conjugates prepared from cross-reacting steroids. There is a dearth of antibodies against cross-reacting steroids, and so one aspect of this invention is the creation of the necessary antibody content in order to cover the cross-reactivity space.

Compounds referred to herein have the following structures:

Another aspect of this invention is the use of multiple fluorophores to introduce additional assay dimensions. In one embodiment a cortisol 3-CMO conjugate with AlexaFluor® 555 dye is employed with a cortisol 21-HS conjugate with AlexaFluor® 647 dye. In another embodiment a conjugate of AlexaFluor® 647 dye and one of the cross-reacting substances could be added to the assay mixture along with the cortisol-AlexaFluor® 555 conjugate. These embodiments would allow for the expansion of the array to include antibodies that may not bind the cortisol 3-CMO conjugate well, but would be useful for the determination of the cross-reactant concentration.

Compounds referred to herein have the following structures:

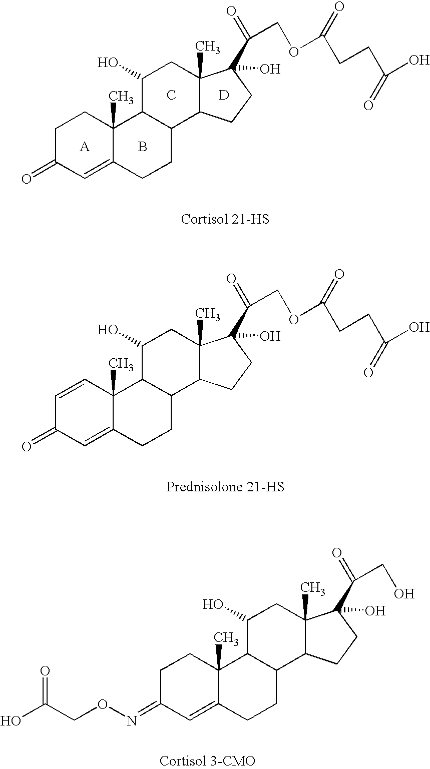

In one embodiment of the present invention, anti-cortisol antibodies are printed in an array on a planar substrate. A conjugate prepared from cortisol and a fluorescent dye is mixed in a buffered solution with a calibrator or a patient sample and then applied to the array of antibodies on the planar substrate. After incubation for a period of time the surface is washed to remove unbound conjugate, and then the fluorescence intensities at each antibody spot are quantitated. The intensities for the various calibrators are used to construct a standard curve from which the apparent cortisol concentrations for the patient samples are determined. These values of apparent cortisol concentrations are inputted into the following algorithms, whereby the true cortisol concentration is calculated.

### Algorithms:

A cross-reaction model is given by:

y=β₁x₁+β₂x₂+ε

This model is used to estimate the cross-reaction percentage. The independent variables, these are x₁ and x₂ in the model are the various concentrations levels, where x₁ is the concentration level of cortisol and x₂ is the concentration level of the cross-reactant (in this case either 6AMP or prednisolone). The dependent variable, i.e. the variable that we are trying to predict, is the “apparent” cortisol concentration, which is estimated from the concentration-signal 4 parameter logistic regression model. We fit this model using standard multiple linear regression methods to estimate β₁ and β₂. These equations are given by,

$${\hat{β}}_{1} = \frac{( \sum {x}_{1 i} {y}_{i} ) ( \sum {x}_{2 i}^{2} ) - ( \sum {x}_{1 i} {x}_{2 i} ) ( \sum {x}_{2 i} {y}_{i} )}{( \sum {x}_{1 i}^{2} ) ( \sum {x}_{2 i}^{2} ) - {( \sum {x}_{1 i} {x}_{2 i} )}^{2}}$$

$${\hat{β}}_{2} = \frac{( \sum {x}_{2 i} {y}_{i} ) ( \sum {x}_{1 i}^{2} ) - ( \sum {x}_{1 i} {x}_{2 i} ) ( \sum {x}_{1 i} {y}_{i} )}{( \sum {x}_{1 i}^{2} ) ( \sum {x}_{2 i}^{2} ) - {( \sum {x}_{1 i} {x}_{2 i} )}^{2}}$$

In terms of the model β₁ is interpreted as the amount that the “apparent” cortisol is increase per ng/ml increase of cortisol and similarly β2 is interpreted as the amount that the “apparent” cortisol is increased per ng/ml increase of cross-reactant. Finally to estimate the percent cross action it is given by:

$$cross reaction percentage = 100 \% * ( \frac{{β}_{2}}{{β}_{1}} )$$

Deconvolution Model:

y=β₁x₁+β₂x₂+β₃x₃+ε

The deconvolution model is used to deconvolute the “apparent” cortisol concentration from the 3 antibodies (Antibody 7, 9 and 10), wherein:

Antibody 7: Clone XM210 from Abcam
Antibody 9: Clone F4P1A3 from EMD Biosciences
Antibody 10: Clone A29220314P from BiosPacific.

There are 3 independent variables: x₁ is the apparent cortisol concentration from antibody 7, x₂ is the apparent cortisol concentration from antibody 9 and x₃ is the apparent cortisol concentration from antibody 10. Here the dependent variable is the “true” or “estimated” cortisol concentration. Here all of the independent variables are data that comes from the concentration signal 4 parameter logistic regression model. The parameters β₁, β₂ and β₃ are estimated using standard multiple linear regression methods.

Interpreting the parameters of the model are done as follows:

$$\frac{\vert {β}_{1} \vert}{\vert {β}_{1} \vert + \vert {β}_{2} \vert + \vert {β}_{3} \vert}$$

is the percentage of “apparent” cortisol of antibody 7 is contributing to the “true” cortisol concentration, similarly,

$$\frac{\vert {β}_{2} \vert}{\vert {β}_{1} \vert + \vert {β}_{2} \vert + \vert {β}_{3} \vert}$$

is the percentage of “apparent” cortisol of antibody 9 is contributing to the “true” cortisol concentration, finally,

$$\frac{\vert {β}_{3} \vert}{\vert {β}_{1} \vert + \vert {β}_{2} \vert + \vert {β}_{3} \vert}$$

is the percentage of “apparent” cortisol of antibody 10 is contributing to the “true” cortisol concentration.

The algorithms, which ultimately involve a regression analysis of multiple variables allow for the determination of true analyte levels in a test sample comprising competitive ligands.

### Particular Aspects of the Invention

One embodiment of the present invention provides a method for determining the concentration of an analyte in a test sample comprising the analyte and a plurality of competitive ligands, the method comprising:

contacting the test sample with at least two different anti-analyte antibodies, wherein each of the antibodies bind the analyte and have a different level of cross-reactivity for the competitive ligands;

detecting binding of the analytes and competitive ligands to the antibodies, thereby determining an observed analyte binding amount for each antibody; and

performing a regression analysis on the observed analyte binding amount for each antibody to determine the concentration of the analyte in the test sample.

In another embodiment of the present invention, the regression analysis is linear regression.

In another embodiment, the regression analysis is non-linear regression.

In another embodiment, the regression analysis is displayed graphically.

In another embodiment, the regression analysis comprises solving the formula:

Y=Σβₙxₙ+c

wherein,

Y is the cortisol concentration;

n is the number of antibodies;

x is the observed steroid amount for each antibody;

β is the level of cross-reactivity for each antibody;

c is a calibration constant; and

Σ is the sum of βx for all antibodies.

In another embodiment, the test sample is plasma, serum, saliva, or urine.

In another embodiment, the analyte is cortisol. More particularly, the competitive ligands are selected from the group consisting of prednisolone, cortisone, 6-a methylprednisolone (6-AMP), progesterone, prednisone, fludrocortisone and dexamethasone.

In another embodiment, the analyte is a drug. In another more particular embodiment thereof, the drug is a structural analogue or derivative of a naturally occurring molecule. More particularly, the drug is a nucleoside analog or a peptide.

In another embodiment, the analyte is prednisolone, dexamethasone, herbicidal triazines, or human T-cell lymphotropic virus (HTLV).

In another embodiment, the test sample is contacted with an analyte comprising a label or a competitive ligand comprising a label prior to the detecting step.

In another embodiment, the label is a fluorescent label.

In another embodiment, the label is an enzyme.

In another embodiment, the label is alkaline phosphotase or horseradish peroxidase (HRP).

In another embodiment, the label comprises a xanthene, an indole, a benzofuran, a cyanine, a coumarin, a borapolyazaindacene, a phycobilliprotein, or a semiconductor nanocrystal.

In another embodiment, the labeled analyte or labeled competitive ligand comprises a label that is bound to the cortisol or prednisolone through a carboxymethyloxime linker.

In another embodiment, the label is bound to the cortisol or prednisolone through a succinate linker.

In another embodiment, the label emits a detectable wavelength which corresponds to a signal intensity.

In another embodiment, the observed analyte binding amount is inversely proportional to the signal intensity.

In another embodiment, the signal intensity from the test sample is compared with an intensity obtained from a sample having a known concentration of analyte and/or competitive ligands.

In another embodiment, the antibodies are monoclonal antibodies.

In another embodiment, the antibodies are polyconal antibodies.

In another embodiment, the antibodies are immobilized on a solid support.

In another embodiment, the solid support is comprised of acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, or polyamino acids.

In another embodiment, the solid support is a bead.

In another embodiment, the solid support further comprises at least one of a thin film, membrane, bottles, dishes, fibers, woven fibers, shaped polymers, particles, beads, or microparticles.

In another embodiment, the method/assay is performed in a buffered solution.

In another embodiment, the regression analysis is performed by a computer.

In another embodiment, the antibodies are produced by immunization of a mammal with a succinate bound analyte.

In another embodiment, the test sample is contacted with at least 1, 2, 3, 4, 5, 6, 7, 8, 9, 10, 11, 12, 13, 14, or 15 antibodies.

In another embodiment, the test sample is contacted with at least 1-3, 2-3, 2-4, 2-5, 2-6, 2-7, 2-8, 2-9, 2-10, 3-4, 3-5, 3-6, 3-7, 3-8, 4-5, 4-6, 4-7, or 5-6 antibodies.

In another embodiment, the test sample is from an individual suspected of having or diagnosed with a disease associated with the analyte. Another embodiment of the present invention provides a compound comprising the structure:

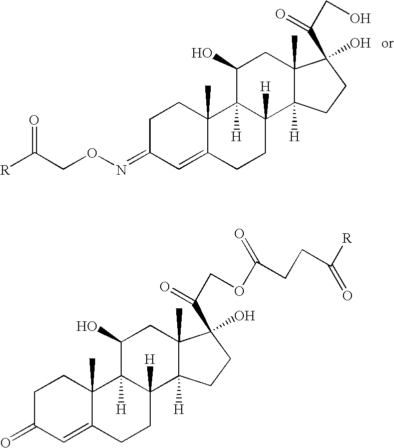

wherein,

R is a label.

Another more particular embodiment thereof provides a composition comprising the compound shown above and at least one antibody that binds the compound.

Another embodiment of the present invention provides a compound comprising the structure:

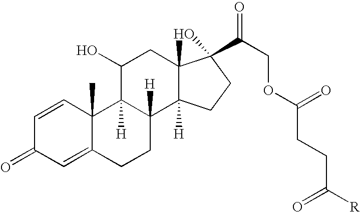

wherein,

R is a label.

Another more particular embodiment thereof provides a composition comprising the compound shown above and at least one antibody that binds the compound.

Another embodiment of the present invention provides a method for determining the concentration of cortisol in a test sample, the method comprising:

contacting the test sample with at least two different anti-steroid antibodies, wherein each of the antibodies bind cortisol and have a different level of cross-reactivity with non-cortisol steroids;

detecting binding of steroids to the antibodies, thereby determining an observed steroid binding amount for each antibody; and

performing a regression analysis on the observed steroid binding amounts for each antibody to determine the concentration of cortisol in the test sample.

In another embodiment, the regression analysis is linear regression.

In another embodiment, the regression analysis is non-linear regression.

In another embodiment, the regression analysis is displayed graphically.

In another embodiment, the regression analysis comprises solving the formula:

Y=Σβₙxₙ+c

wherein,

Y is the cortisol concentration;

n is the number of antibodies;

x is the observed steroid amount for each antibody;

β is the level of cross-reactivity for each antibody;

c is a calibration constant; and

Σ is the sum of βx for all antibodies.

In another embodiment, the test sample is plasma, serum, saliva, or urine.

In another embodiment, the analyte or non-cortisol steroid is selected from the group consisting of Betamethasone, Budesonide, Cortisone, Dexamethasone, Hydrocortisone, Methylprednisolone, Prednisolone, Prednisone, and Triamcinolone.

In another embodiment, the non-cortisol steroids are selected from the group consisting of prednisolone, cortisone, 6-α methylprednisolone (6-AMP), progesterone, prednisone, fludrocortisone and dexamethasone.

In another embodiment, the test sample is contacted with cortisol comprising a label or prednisolone comprising a label prior to the detecting step.

In another embodiment, the label is a fluorescent label.

In another embodiment, the label is an enzyme.

In another embodiment, the label is alkaline phosphotase or horseradish peroxidase (HRP).

In another embodiment, the label comprises a xanthene, an indole, a benzofuran, a cyanine, a coumarin, a borapolyazaindacene, a phycobilliprotein, or a semiconductor nanocrystal.

In another embodiment, the label is bound to the cortisol or prednisolone through a carboxymethyloxime linker.

In another embodiment, the label is bound to the cortisol or prednisolone through a succinate linker.

In another embodiment, the cortisol comprising a label is:

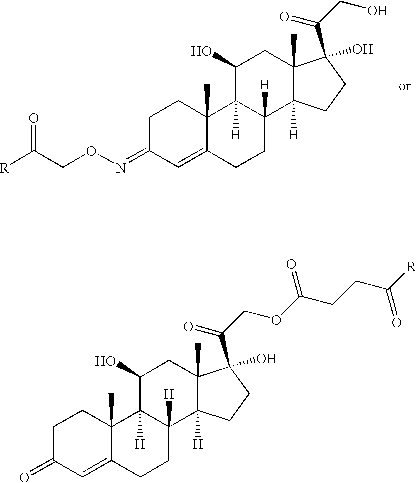

wherein,

R is a label.

In another embodiment, the prednisolone comprising a label is:

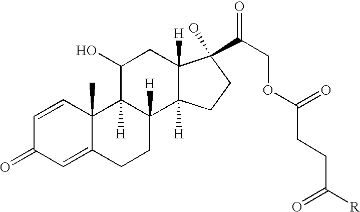

wherein,

R is a label.

In another embodiment, the label emits a detectable wavelength which corresponds to a signal intensity.

In another embodiment, the observed steroid binding amount is inversely proportional to the signal intensity.

In another embodiment, the signal intensity from the test sample is compared with an intensity obtained from a control sample having a known concentration of steroids.

In another embodiment, the antibodies are monoclonal antibodies.

In another embodiment, the antibodies are polyconal antibodies.

In another embodiment, the antibodies are immobilized on a solid support.

In another embodiment, the solid support is comprised of acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, or polyamino acids.

In another embodiment, the solid support is a bead.

In another embodiment, the solid support further comprises at least one of a thin film, membrane, bottles, dishes, fibers, woven fibers, shaped polymers, particles, beads, or microparticles.

In another embodiment, the contacting step is performed in a buffered solution.

In another embodiment, the regression analysis is performed by a computer.

In another embodiment, the anti-steroid antibodies are produced by immunization of a mammal with a succinate bound steroid.

In another embodiment, the test sample is contacted with at least three antibodies.

In another embodiment, the test sample is contacted with at least five antibodies.

In another embodiment, the test sample is contacted with at least 1, 2, 3, 4, 5, 6, 7, 8, 9, 10, 11, 12, 13, 14, or 15 antibodies.

In another embodiment, the test sample is contacted with 1-3, 2-3, 2-4, 2-5, 2-6, 2-7, 2-8, 2-9, 2-10, 3-4, 3-5, 3-6, 3-7, 3-8, 4-5, 4-6, 4-7, or 5-6 antibodies.

In another embodiment, the test sample is from an individual suspected of having or diagnosed with Cushing's syndrome or Addison's disease.

In another embodiment, the test sample is from an individual receiving treatment to modulate cortisol levels.

In another embodiment, the treatment comprises administration of hydrocortisone, Prednisone or Relacore.

In another embodiment, the test sample is from an individual that has hypercortisolism or hypocortisolism.

Another embodiment of the present invention provides a composition comprising at least three different isolated anti-steroid antibodies, wherein each of the antibodies bind cortisol and have a different level of cross-reactivity with non-cortisol steroids.

Another embodiment thereof further comprises a test sample from an individual.

In another embodiment, the antibodies are present in admixture.

In another embodiment, the antibodies are immobilized on a solid support.

In another embodiment, the solid support is comprised of acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, or polyamino acids.

In another embodiment, the solid support is a bead.

In another embodiment, the solid support further comprises at least one of a thin film, membrane, bottles, dishes, fibers, woven fibers, shaped polymers, particles, beads, or microparticles.

In another embodiment, the test sample is plasma, serum, saliva, or urine.

Another embodiment thereof further comprises labeled cortisol or labeled prednisolone.

In another embodiment, the label is a fluorescent label.

In another embodiment, the label is an enzyme.

In another embodiment, the label is alkaline phosphotase or horseradish peroxidase (HRP).

In another embodiment, the label comprises a xanthene, an indole, a benzofuran, a cyanine, a coumarin, a borapolyazaindacene, a phycobilliprotein, or a semiconductor nanocrystal.

In another embodiment, the label is bound to the cortisol or prednisolone through a carboxymethyloxime linker.

In another embodiment, the label is bound to the cortisol or prednisolone through a succinate linker.

In another embodiment, the cortisol comprising a label is:

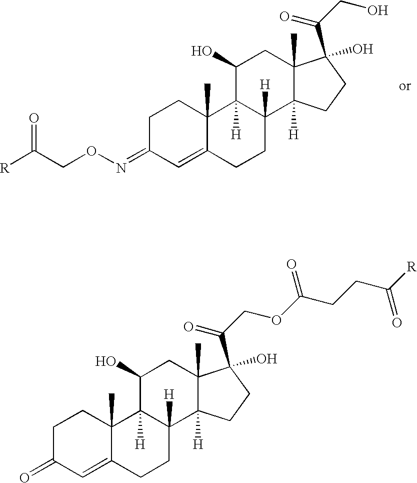

wherein,

R is a label.

In another embodiment, the prednisolone comprising a label is:

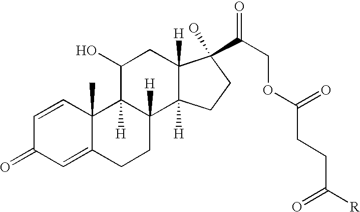

wherein,

R is a label.

In another embodiment, the label emits a detectable wavelength which corresponds to a signal intensity.

In another embodiment, the antibodies are monoclonal antibodies.

In another embodiment, the antibodies are polyconal antibodies.

In another embodiment, the composition comprises at least 2, 3, 4, 5, 6, 7, 8, 9, 10, 11, 12, 13, 14, or 15 anti-steroid antibodies, wherein each of the antibodies bind cortisol and have a different level of cross-reactivity with non-cortisol steroids.

In another embodiment, the array comprises 2-3, 2-4, 2-5, 2-6, 2-7, 2-8, 2-9, 2-10, 3-4, 3-5, 3-6, 3-7, 3-8, 4-5, 4-6, 4-7, or 5-6 antibodies.

Another embodiment of the present invention provides an array device comprising a solid support comprising at least two different anti-steroid antibodies, wherein each of the antibodies bind cortisol and have a different level of cross-reactivity with non-cortisol steroids.

In another embodiment, the solid support is comprised of acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, or polyamino acids.

In another embodiment, the array further comprises at least one of a thin film, membrane, bottles, dishes, fibers, woven fibers, shaped polymers, particles, beads, or microparticles.

Another embodiment thereof further comprises a test sample from an individual.

In another embodiment, the test sample is plasma, serum, saliva, or urine.

Another embodiment of the array further comprises labeled cortisol or labeled prednisolone in contact with the solid support.

In another embodiment, the label is a fluorescent label.

In another embodiment, the label is an enzyme.

In another embodiment, the label is alkaline phosphotase or horseradish peroxidase (HRP).

In another embodiment, the label comprises a xanthene, an indole, a benzofuran, a cyanine, a coumarin, a borapolyazaindacene, a phycobilliprotein, or a semiconductor nanocrystal.

In another embodiment, the label is bound to the cortisol or prednisolone through a carboxymethyloxime linker.

In another embodiment, the label is bound to the cortisol or prednisolone through a succinate linker.

In another embodiment, the cortisol comprising a label is:

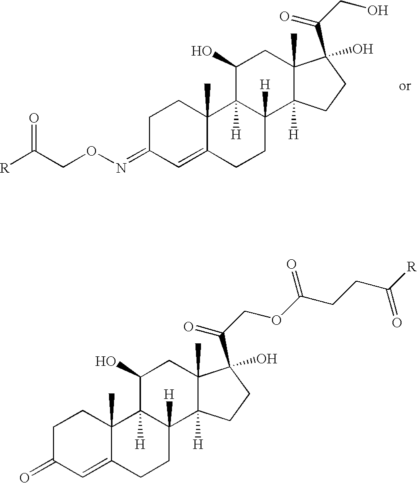

wherein,

R is a label.

In another embodiment, the prednisolone comprising a label is:

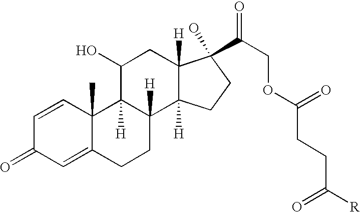

wherein,

R is a label.

In another embodiment, the label emits a detectable wavelength which corresponds to a signal intensity.

In another embodiment, the antibodies are monoclonal antibodies.

In another embodiment, the antibodies are polyconal antibodies.

In another embodiment, the array comprises at least 2, 3, 4, 5, 6, 7, 8, 9, 10, 11, 12, 13, 14, or 15 anti-steroid antibodies, wherein each of the antibodies bind cortisol and have a different level of cross-reactivity with non-cortisol steroids.

In another embodiment, the composition comprises 2-3, 2-4, 2-5, 2-6, 2-7, 2-8, 2-9, 2-10, 3-4, 3-5, 3-6, 3-7, 3-8, 4-5, 4-6, 4-7, or 5-6 antibodies.

Another embodiment of the present invention provides a kit for determining cortisol concentration in a test sample comprising:

a solid support comprising at least two different anti-steroid antibodies, wherein each of the antibodies bind cortisol and have a different level of cross-reactivity with non-cortisol steroids; and

instructions on how to determine the cortisol concentration in the test sample.

Another embodiment of the kit further comprises a buffer solution.

Another embodiment of the kit involves a test sample.

Another embodiment of the kit involves a control sample. The control sample can have a predetermined amount of analyte, such as cortisol.

Another embodiment of the kit further comprises a computer for performing calculations to determine the cortisol concentration in the test sample. More particularly, the calculations comprise a linear regression analysis.

In another embodiment, the solid support is comprised of acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, or polyamino acids.

In another embodiment, the kit further comprises at least one of a thin film, membrane, bottles, dishes, fibers, woven fibers, shaped polymers, particles, beads, or microparticles.

Another embodiment of the kit further comprises a test sample from an individual.

In another embodiment, the test sample is plasma, serum, saliva, or urine.

Another embodiment of the kit further comprises labeled cortisol or labeled prednisolone in contact with the solid support.

In another embodiment, the label is a fluorescent label.

In another embodiment, the label is an enzyme.

In another embodiment, the label is alkaline phosphotase or horseradish peroxidase (HRP).

In another embodiment, the label comprises a xanthene, an indole, a benzofuran, a cyanine, a coumarin, a borapolyazaindacene, a phycobilliprotein, or a semiconductor nanocrystal.

In another embodiment, the label is bound to the cortisol or prednisolone through a carboxymethyloxime linker.

In another embodiment, the label is bound to the cortisol or prednisolone through a succinate linker.

In another embodiment, the cortisol comprising a label is:

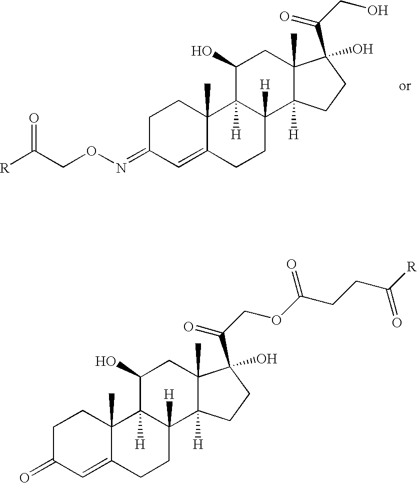

wherein,

R is a label.

In another embodiment, the prednisolone comprising a label is:

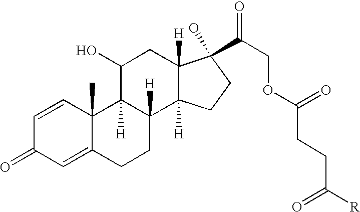

wherein,

R is a label.

In another embodiment, the label emits a detectable wavelength which corresponds to a signal intensity.

In another embodiment, the antibodies are monoclonal antibodies.

In another embodiment, the antibodies are polyconal antibodies.

In another embodiment, the kit comprises at least 2, 3, 4, 5, 6, 7, 8, 9, 10, 11, 12, 13, 14, or 15 anti-steroid antibodies, wherein each of the antibodies bind cortisol and have a different level of cross-reactivity with non-cortisol steroids.

In another embodiment, the kit comprises 2-3, 2-4, 2-5, 2-6, 2-7, 2-8, 2-9, 2-10, 3-4, 3-5, 3-6, 3-7, 3-8, 4-5, 4-6, 4-7, or 5-6 antibodies.

Another embodiment of the present invention provides a method for detecting cortisol levels in an individual, the method comprising:

contacting a test sample from the individual with at least two different anti-steroid antibodies, wherein each of the antibodies bind cortisol and have a different level of cross-reactivity with non-cortisol steroids;

detecting binding of steroids to each of the antibodies, thereby determining an observed steroid binding amount for each of the antibodies;

performing a regression analysis on the observed steroid binding amounts for each antibody to determine the concentration of cortisol in the test sample; and

comparing the concentration of cortisol in the test sample from the individual with cortisol levels in a control sample to detect cotisol levels in the individual.

In another embodiment, the regression analysis is linear regression.

In another embodiment, the regression analysis is non-linear regression.

In another embodiment, the regression analysis is displayed graphically.

In another embodiment, the regression analysis comprises solving the formula:

Y=Σβₙxₙ+c

wherein,

Y is the cortisol concentration;

n is the number of antibodies;

x is the observed steroid amount for each antibody;

β is the level of cross-reactivity for each antibody;

c is a calibration constant; and

Σ is the sum of βx for all antibodies.

In another embodiment, the test sample is plasma, serum, saliva, or urine.

In another embodiment, the non-cortisol steroids are selected from the group consisting of prednisolone, cortisone, 6-a methylprednisolone (6-AMP), progesterone, prednisone, fludrocortisone and dexamethasone.

In another embodiment, the test sample is contacted with cortisol comprising a label or prednisolone comprising a label prior to the detecting step.

In another embodiment, the label is a fluorescent label.

In another embodiment, the label is an enzyme.

In another embodiment, the label is alkaline phosphotase or horseradish peroxidase (HRP).

In another embodiment, the label comprises a xanthene, an indole, a benzofuran, a cyanine, a coumarin, a borapolyazaindacene, a phycobilliprotein, or a semiconductor nanocrystal.

In another embodiment, the label is bound to the cortisol or prednisolone through a carboxymethyloxime linker.

In another embodiment, the label is bound to the cortisol or prednisolone through a succinate linker.

In another embodiment, the cortisol comprising a label is:

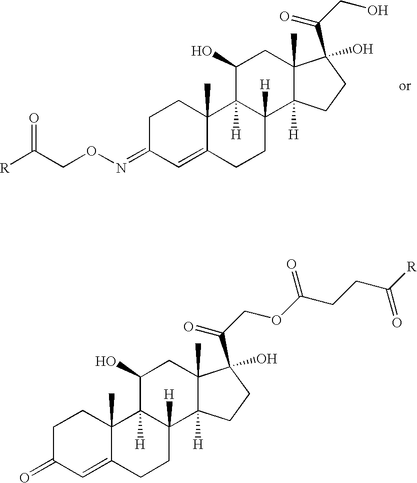

wherein,

R is a label.

In another embodiment, the prednisolone comprising a label is:

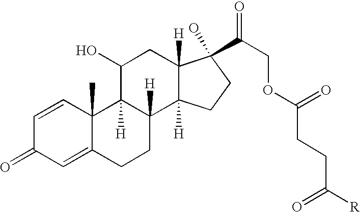

wherein,

R is a label.

In another embodiment, the label emits a detectable wavelength which corresponds to a signal intensity.

In another embodiment, the observed steroid binding amount is inversely proportional to the signal intensity.

In another embodiment, the signal intensity from the test sample is compared with an intensity obtained from a control sample having a known concentration of steroids.

In another embodiment, the antibodies are monoclonal antibodies.

In another embodiment, the antibodies are polyconal antibodies.

In another embodiment, the antibodies are immobilized on a solid support.

In another embodiment, the solid support is comprised of acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, or polyamino acids.

In another embodiment, the solid support is a bead.

In another embodiment, the solid support further comprises at least one of a thin film, membrane, bottles, dishes, fibers, woven fibers, shaped polymers, particles, beads, or microparticles.

In another embodiment, the contacting step is performed in a buffered solution.

In another embodiment, the regression analysis is performed by a computer.

In another embodiment, the anti-steroid antibodies are produced by immunization of a mammal with a succinate bound steroid.

In another embodiment, the test sample is contacted with at least three antibodies.

In another embodiment, the test sample is contacted with at least five antibodies.

In another embodiment, the test sample is contacted with at least 1, 2, 3, 4, 5, 6, 7, 8, 9, 10, 11, 12, 13, 14, or 15 antibodies.

In another embodiment, the test sample is contacted with 1-3, 2-3, 2-4, 2-5, 2-6, 2-7, 2-8, 2-9, 2-10, 3-4, 3-5, 3-6, 3-7, 3-8, 4-5, 4-6, 4-7, or 5-6 antibodies.

In another embodiment, the test sample is from an individual suspected of having or diagnosed with Cushing's syndrome or Addison's disease.

In another embodiment, the test sample is from an individual receiving treatment to modulate cortisol levels.

In another embodiment, the treatment comprises administration of hydrocortisone, Prednisone or Relacore.

In another embodiment, the test sample is from an individual that has hypercortisolism or hyporcortisolism.

Another embodiment of the present invention provides a method of using a computer processor to determine the concentration of cortisol in a test sample, the method comprising:

receiving data representing observed steroid concentrations in a test sample, wherein the data is obtained from contacting at least two different anti-steroid antibodies with a test sample, wherein each of the antibodies bind cortisol and have a different level of cross-reactivity with non-cortisol steroids; and

performing a linear regression analysis with the computer processor with the data to determine a result comprising the concentration of cortisol in the test sample.

In another embodiment, the regression analysis is linear regression.

In another embodiment, the regression analysis is non-linear regression.

In another embodiment, the result is displayed graphically.

In another embodiment, the regression analysis comprises solving the formula:

Y=Σβₙxₙ+c

wherein,

Y is the cortisol concentration;

n is the number of antibodies;

x is the observed steroid amount for each antibody;

β is the level of cross-reactivity for each antibody;

c is a calibration constant; and

Σ is the sum of βx for all antibodies.

In another embodiment, the test sample is plasma, serum, saliva, or urine.

In another embodiment, the non-cortisol steroids are selected from the group consisting of prednisolone, cortisone, 6-α methylprednisolone (6-AMP), progesterone, prednisone, fludrocortisone and dexamethasone.

In another embodiment, the test sample is contacted with cortisol comprising a label or prednisolone comprising a label prior to the detecting step.

In another embodiment, the label is a fluorescent label.

In another embodiment, the label is an enzyme.

In another embodiment, the label is alkaline phosphotase or horseradish peroxidase (HRP).

In another embodiment, the label comprises a xanthene, an indole, a benzofuran, a cyanine, a coumarin, a borapolyazaindacene, a phycobilliprotein, or a semiconductor nanocrystal.

In another embodiment, the label is bound to the cortisol or prednisolone through a carboxymethyloxime linker.

In another embodiment, the label is bound to the cortisol or prednisolone through a succinate linker.

In another embodiment, the cortisol comprising a label is:

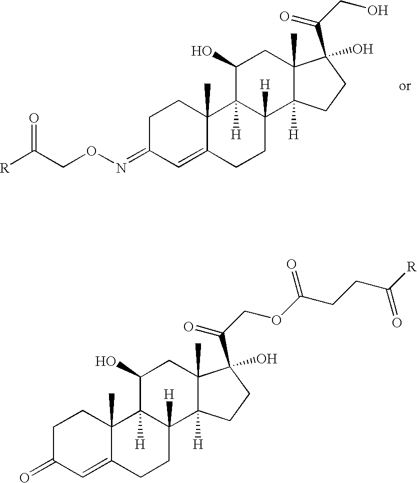

wherein,

R is a label.

In another embodiment, the prednisolone comprising a label is:

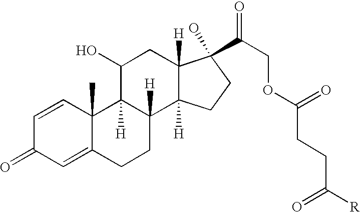

wherein,

R is a label.

In another embodiment, the label emits a detectable wavelength which corresponds to a signal intensity.

In another embodiment, the observed steroid binding amount is inversely proportional to the signal intensity.

In another embodiment, the signal intensity from the test sample is compared with an intensity obtained from a control sample having a known concentration of steroids.

In another embodiment, the antibodies are monoclonal antibodies.

In another embodiment, the antibodies are polyconal antibodies.

In another embodiment, the antibodies are immobilized on a solid support.

In another embodiment, the solid support is comprised of acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, or polyamino acids.

In another embodiment, the solid support is a bead.

In another embodiment, the solid support further comprises at least one of a thin film, membrane, bottles, dishes, fibers, woven fibers, shaped polymers, particles, beads, or microparticles.

In another embodiment, the contacting step is performed in a buffered solution.

In another embodiment, the regression analysis is performed using Microsoft® Excel software.

In another embodiment, the anti-steroid antibodies are produced by immunization of a mammal with a succinate bound steroid.

In another embodiment, the test sample is contacted with at least three antibodies.

In another embodiment, the test sample is contacted with at least five antibodies.

In another embodiment, the test sample is contacted with at least 1, 2, 3, 4, 5, 6, 7, 8, 9, 10, 11, 12, 13, 14, or 15 antibodies.

In another embodiment, the test sample is contacted with 1-3, 2-3, 2-4, 2-5, 2-6, 2-7, 2-8, 2-9, 2-10, 3-4, 3-5, 3-6, 3-7, 3-8, 4-5, 4-6, 4-7, or 5-6 antibodies.

In another embodiment, the test sample is from an individual suspected of having or diagnosed with Cushing's syndrome or Addison's disease.

In another embodiment, the test sample is from an individual receiving treatment to modulate cortisol levels.

In another embodiment, the treatment comprises administration of hydrocortisone, Prednisone or Relacore.

In another embodiment, the test sample is from an individual that has hypercortisolism or hyporcortisolism.

Additional aspects of the invention include any combination of the aforementioned embodiments.

The present invention will be understood more readily by reference to the following examples, which are provided by way of illustration and are not intended to be limiting of the present invention.

### EXAMPLES

### I. Synthesis of Cortisol Alexa Fluor Conjugates

Carboxylic acid derivatives of cortisol and prednisolone were used to prepare the Alexa Fluor conjugates. Cortisol 3-carboxymethyl oxime (3-CMO), cortisol 21-hemisuccinate (21-HS), and prednisolone 21-HS were activated with EDC and N-hydroxysuccinimide in DMF. The steroid active ester was reacted with the cadaverine derivatives of Alexa-Fluor dyes. The steroid Alexa-Fluor conjugates were purified by HPLC using a Zorbax C-18 column in 100 mM triethyl ammonium acetate pH 7 with gradient elution by acetonitrile.

### II. Synthesis of BSA-cortisol 21-HS and BSA-prednisolone 21-HS

Ten milligrams of steroid carboxylic acid (cortisol 21-hemisuccinate or prednisolone 21-hemisuccinate) was dissolved in 0.05 mL of dimethylformamide (DMF). To this solution was added 0.026 mL of 100 mg/mL N-hydroxysuccinimide (NHS) followed by 0.176 mL of 25 mg/mL of N-(3-Dimethylaminopropyl)-N′-ethylcarbodiimide hydrochloride (EDC). The reaction proceeded at room temperature for 2.5 hours. The steroid NHS ester reaction mixture was added to 5 mL of a 10 mg/mL solution of bovine serum albumin (BSA) in bicarbonate-buffered saline, pH 8.6, at a stoichiometry of 50 mol steroid-NHS ester per 1 mol BSA. The reaction proceeded at room temperature for 2.5 hours then stored at 4° C. for 2 days. The steroid-BSA conjugates were purified by gel filtration chromatography on Sephadex G-25 in phosphate-buffered saline (PBS).

### III. Preparation of Anti steroid Antibodies

The steroid-BSA derivatives were used to immunize rabbits in order to generate antibodies against the 21-hemisuccinate derivatives of the steroids cortisol and prednisolone. The IgG fractions from the antisera were first purified on Protein A Sepharose beads followed by affinity purification of the specific anti-steroid IgG on Sepharose beads that contained the 21-hemisuccinate derivatives of cortisol or prednisolone covalently linked to the bead surface. The bound antibody fraction was eluted with glycine buffer at pH 2.5, and the pH was immediately neutralized with TRIS. The affinity-purified polyclonal antibodies were dialyzed against PBS.

### IV. Antibody Micro-Array Printing

Anti cortisol and prednisolone antibodies (ones that are commercially available and those generated in house) were printed on 25×75 mm glass slides that had a silyl-epoxy coating (Telechem, Super Epoxy 2). The antibody concentrations were 125 ug/mL in buffered solutions comprising phosphate-buffered saline (PBS) and 1× Whatman Protein Arraying Buffer. The antibody solutions were applied to the slide surface using a Scienion sciflexarryer S5 piezo printer. The average spot sizes were 150 um. The antibodies within a sub array were printed in replicates of five spots, and each slide contained twelve sub arrays arranged in two columns of six. The relative spacing of the sub arrays was nine millimeters, equivalent to the spacing of a 96-well micro titer plate.

### V. Assays of Cortisol-Containing Samples

The micro-array slides were assembled with a superstructure that creates individual wells that surround the sub array (Slide Incubation Chambers, Whatman). The slides were blocked for one hour with micro array blocking buffer (VWR) and then washed with TRIS-buffered saline with Tween-20 (TBST). Cortisol-containing samples or calibrators were diluted 1 to 20 into a buffered solution that contained the following: TBST, 0.1% SDS, 3.3 nM cortisol 3-CMO Alexa Fluor 555, and 3.3 nM cortisol 21-HS Alexa Fluor 647. One-hundred micro liters of the assay mixture was applied to an individual well on the micro-array slide, and the assay was allowed to proceed for one hour at room temperature in the dark with gentle shaking on a micro-titer plate shaker. The assay mixture was removed, and the slides were washed with TBST, water, and then dried by spinning in a centrifuge. The intensities of fluorescence associated with each spot was quantified using a micro-array reader such as an Axon 4000B or 4200AL. The spot intensities for the calibrators were used to generate a standard curve by fitting the data to a four-parameter logistic model. The concentrations of the cortisol-containing patient samples or synthetic samples were interpolated using the logistic-fit equation.

### VI. Multiple Regression Analysis

The apparent cortisol concentrations obtained on each different antibody spot in the micro array were used to determine the regression coefficients for a linear equation. A training set of data was generated using samples that contained known concentrations of cortisol and spiked cross-reacting steroids. The true concentrations of cortisol were defined as the dependent variables and the apparent cortisol concentrations determined at each unique antibody spot in the micro array were defined as the independent variables. A multiple-regression analysis was performed using Microsoft Excel, and the coefficients were determined, using the following equation as the model:

y=β₁x_{1+β}₂x₂+ . . . +βₙxₙ+c

where y is the true cortisol concentration, xₙ is the apparent cortisol concentration for a given antibody in the micro array, βᵢ is the regression coefficient for the respective antibody, and c is a constant. The same mathematical model is used to calculate the true cortisol concentration for an unknown sample. The previously determined β values that had p values <0.05 and the measured apparent cortisol concentrations obtained from the micro array analysis of an unknown sample are combined to yield the true cortisol concentration.

The above model can then be generalized to a general linear model where the relationship between the true cortisol and the apparent coritsol concentrations is given by,

y=g(β₁x₁+β₂x₂+ . . . +βₙxₙ)+c

where g is known as the link function, and y, βᵢ, xᵢ and c have the same definition. Additionally any polynomial regression function given by,

y=β_{1,1}x_{1+β}_{1,2}x₁² . . . β_{1,k1}x₁^{k1}+β_{2,1}x₂+β_{2,2}x₂² . . . +β_{2,k2}x₂^{k2} . . . +β_{n,1}xₙ+β_{n,1}xₙ+β_{n,2}xₙ² . . . β_{n,kn}xₙ^{kn}+c

where y, xᵢ and c have the same interpretation as before, and β_{i,ki} is the regression coefficient for the i^{th} antibody for the ki^{th} polynomial term.

### VII. Antibody Chip Preparation

Arrays of anti-cortisol antibodies having different cross-relativities are printed on epoxy-functionalized glass slides. A contact printing robot (PixSys 5500; Cartesian Technologies) with a stealth microspotting pin (Model SMP4; TeleChem International) is used to print the antibodies on the epoxy-functionalized glass slides. The concentration each the printed antibody (anti-cortisol) is 125 mg/L in Protein Printing Buffer (Whatman). The antibody is reacted on the protein chip for 6 h in a humidified chamber. The slide is then stored at room temperature for up to 1 month.

### VIII. Immunoassay Procedure

A competitive immunoassay design is used to test patient samples for cortisol levels. A molded polyester frame is attached to the substrate to partition 12 arrays on the antibody chip surface. This protein chip consists of multiple different ant-cortisol antibodies having varying cross-reactivity for other close structural cortisol analogues. The antibody chips are blocked in microarray blocking buffer (VWR) for 30 min at room temperature and then rinsed three times with TRIS-buffered saline containing 0.5 mL/L Tween 20, pH 7.4 (TBS-Tween A). A mixture of the fluorescently labeled cortisol and a patient sample (blood, saliva or urine) or control is then applied to the gridded reaction chamber formed by the polyester frame covering the surface of the antibody chip. The antibody chip is then maintained at room temperature with gentle shaking for 60 min. The chip is then rinsed three times with TBS Tween A. The protein chip is subsequently scanned for fluorescently labeled cortisol by use of a laser confocal scanner or a charge coupled device-based scanner. The analog fluorescent signal is converted to digital signal by data analysis software (ArrayVision GE Healthcare; GenePix Pro 4.1; Molecular Devices).

### IX. Assay and Results

Experiments were conducted with the following samples:
0 ng/mL of cortisol, 0 ng/ml of prednisolone
10 ng/mL of cortisol, 0 ng/ml of prednisolone
25 ng/mL of cortisol, 0 ng/ml of prednisolone
53 ng/mL of cortisol, 0 ng/ml of prednisolone
130 ng/mL of cortisol, 0 ng/ml of prednisolone
316 ng/mL of cortisol, 0 ng/ml of prednisolone
0 ng/mL of cortisol, 250 ng/ml of prednisolone
10 ng/mL of cortisol, 250 ng/ml of prednisolone
25 ng/mL of cortisol, 250 ng/ml of prednisolone
53 ng/mL of cortisol, 250 ng/ml of prednisolone
130 ng/mL of cortisol, 250 ng/ml of prednisolone
316 ng/mL of cortisol, 250 ng/ml of prednisolone
0 ng/mL of cortisol, 500 ng/ml of prednisolone
10 ng/mL of cortisol, 500 ng/ml of prednisolone
25 ng/mL of cortisol, 500 ng/ml of prednisolone
53 ng/mL of cortisol, 500 ng/ml of prednisolone
130 ng/mL of cortisol, 500 ng/ml of prednisolone
316 ng/mL of cortisol, 500 ng/ml of prednisolone
0 ng/mL of cortisol, 1000 ng/ml of prednisolone
10 ng/mL of cortisol, 1000 ng/ml of prednisolone
25 ng/mL of cortisol, 1000 ng/ml of prednisolone
53 ng/mL of cortisol, 1000 ng/ml of prednisolone
130 ng/mL of cortisol, 1000 ng/ml of prednisolone
316 ng/mL of cortisol, 1000 ng/ml of prednisolone

Each sample was tested on 6 different antibodies (denoted as antibody 7, 9, 10, 39, 40 and 41), defined above. For each antibody a competitive assay is done to known concentration samples measured in relation to signal measured, wherein the results are modeled with a 4 parameter logistic function. With the concentration of samples known, the parameters of the model are estimated. With the estimated model parameters, the concentrations of the given samples for each of the 6 antibodies is estimated. A multiple regression model is built to estimate true cortisol concentrations from the estimated concentrations from the 6 individual antibodies (a backwards regression model fitting from a saturated model to a reduced model is performed as shown in Table 6). Hence for each sample there are six different estimates of concentration, antibodies 1-6 and model estimate.

Initial synthetic glucocorticoid antibody cross reactivity data included LC-MS/MS analysis of patient samples was followed by analysis via the Beckman immunoassay system, and relative cross reactivities were calculated for each of the synthetic glucocorticoids of interest. Cross reactivities were also calculated based on a linearity study of each of the major cross reactive synthetic glucocorticoids to ensure that the values obtained for each were viable. (Table 1).

TABLE 1
Synthetic Glucocorticoid cross reactivities in Beckmann Access

| | | Access | | |
|---|---|---|---|---|
| | | Cortisol | | |
| | | Value | % Reactivity | % Reactivity |
| SYN-1 | 6-a-methyl | 34.4 | 1.72 | 1.4 |
| | prednisolone | | | |
| SYN-2 | Prednisone | 50.8 | 2.54 | 2.2 |
| SYN-3 | Triamcinolone | 0 | 0 | NA |
| | acetonide | | | |
| SYN-4 | Triamcinolone | 1.1 | 0.055 | NA |
| SYN-5 | Fludrocortisone | 26.2 | 1.31 | 1.3 |
| SYN-6 | Cortisone | 94 | 4.7 | 4.5 |
| SYN-7 | Prednisolone | 420.7 | 21.035 | 20.8 |
| SYN-8 | Fluorometholone | 0.8 | 0.04 | NA |
| SYN-9 | Betamethasone | 0.8 | 0.04 | NA |
| SYN-10 | Dexamethasone | 0.5 | 0.025 | NA |

Method 1:
All synthetics present at 2000 ng/mL
% Reactivity = (cortisol value)/(concentration of analyte) × 100%
Method 2:
Serial dilutions 1:2, 1:4, 1:8 and 1:16. Linear analysis evaluation

A population was drawn of positive synthetic patient samples once cross reactivities were determined for individual steroids (Table 2).

TABLE 2
Syntetic positives population

| Steroid | Number of | Range (ng/mL) |
|---|---|---|
| Dexamethasone | 32 | 1.0-14.0 |
| Triamcinolone Acetonide | 13 | 0.3-2.3 |
| Prednisolone | 9 | 0.6-640 |
| Prednisone | 5 | 1.9-72 |
| 6a-methylprednisolone | 4 | 1.7-130 |

LC-MS/MS analysis of patient samples was followed by analysis using the Beckman Access (commercial assay), and relative cross reactivities were calculated for each of the synthetic glucocorticoids of interest. Synthetic positive patient samples were then mimicked in stripped serum for cortisol, cortisone and exogenous synthetic glucocorticoids. Final cortisol concentrations were backed out based on cross reactivities and relative concentrations to prove the contribution of the synthetic glucocorticoids to the final observed concentrations observed in immunoassay (Table 3). The linear relationship for these systems was examined and illustrated. The mimic study proved the major contributing factors were indeed the endogenous synthetic glucocorticoids.

TABLE 3
Table 3. Synthetic Glucocorticoid patient mimics.

| | | LC-MS/MS | | DXI | Calculated |
|---|---|---|---|---|---|
| Sample # | Synthetic Present | Cortisol | Cortisone | Cortisol | Cortisol |
| 1 | POSITIVE FOR PREDNISOLONE: 4.9 ng/mL | 3.3 | 0.6 | 7.4 | 4.4 |
| 2 | POSITIVE FOR PREDNISOLONE 450 ng/mL | 7.4 | No Peak | 114.4 | 103.0 |
| | POSITIVE FOR PREDNISONE: 51 ng/mL | | | | |
| 3 | POSITIVE FOR TRIAMCINOLONE ACETONIDE 3.2 ng/mL | 1.1 | 0.2 | 2.6 | 1.1 |
| 4 | POSITIVE FOR DEXAMETHASONE: 3.5 ng/mL | 79.4 | 21.5 | 88.4 | 80.4 |
| 5 | POSITIVE FOR DEXAMETHASONE: 2.4 ng/mL | 37.8 | 6.8 | 43.3 | 38.1 |
| 6 | POSITIVE FOR PREDNISOLONE 8.8 ng/mL | 2.6 | 2.3 | 8.8 | 4.7 |
| | POSITIVE FOR PREDNISONE: 5.9 ng/mL | | | | |
| 7 | POSITIVE FOR DEXAMETHASONE: 2.0 ng/mL | 54.5 | 2.3 | 69.3 | 54.6 |
| 8 | POSITIVE FOR DEXAMTHASONE: 0.8 ng/mL | 25.5 | 4.0 | 29.8 | 25.6 |
| 9 | POSITIVE FOR DEXAMETHASONE: 5.2 ng/mL | 103.0 | 18.3 | 136.1 | 103.9 |
| 10 | POSITIVE FOR TRIAMCINOLONE ACETONIDE 1.9 ng/mL | 8.3 | 1.1 | 12.5 | 8.4 |
| 11 | POSITIVE FOR 6α METHYL PREDNISOLONE 1.7 ng/mL | 5.8 | 1.0 | 7.3 | 5.9 |
| 12 | POSITIVE FOR TRIAMCINOLONE ACETONIDE 2.2 ng/mL | 14.3 | 1.4 | 7.1 | 14.4 |
| 13 | POSITIVE FOR DEXAMETHASONE: 3.7 ng/mL | 4.2 | 0.5 | 3.0 | 4.2 |
| 14 | POSITIVE FOR PREDNISOLONE 640 ng/mL | 18.3 | No Peak | 182.7 | 154.5 |
| | POSITIVE FOR PREDNISONE: 72 ng/mL | | | | |
| 15 | POSITIVE FOR DEXAMETHASONE: 3.8 ng/mL | 8.0 | 2.6 | 10.1 | 8.1 |
| 16 | POSITIVE FOR DEXAMETHASONE: 1.9 ng/mL | 5.8 | 1.0 | 2.8 | 5.8 |
| 17 | POSITIVE FOR TRIAMCINOLONE ACETONIDE 0.4 ng/mL | 4.1 | 1.0 | 1.1 | 4.1 |
| 18 | POSITIVE FOR DEXAMETHASONE: 1.8 ng/mL | 17.7 | 3.9 | 18.7 | 17.9 |
| 19 | POSITIVE FOR PREDNISOLONE 280 ng/mL | 0.0 | No Peak | 51.8 | 59.5 |
| | POSITIVE FOR PREDNISONE: 29 ng/mL | | | | |
| 20 | POSITIVE FOR DEXAMETHASONE: 13.0 ng/mL | 9.4 | 1.0 | 7.9 | 9.5 |
| 21 | POSITIVE FOR DEXAMETHASONE: 2.4 ng/mL | 12.2 | No Peak | 11.5 | 12.2 |

*All concentrations are reported in ng/mL

Subsequently, several cortisol antibodies were synthesized and relative cross reactivities were determined for the synthetic glucocorticoids obtained via the same described the Beckmann system (Tables 4 & 5). Cortisol antibodies should have specificity to distinguish the differences in the A ring of the steroid molecules shown below.

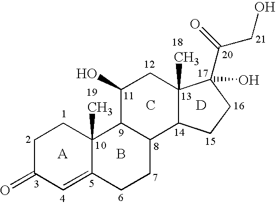

Specificity on the D ring may not allow the antibody to distinguish between cortisol and the synthetic glucocorticoids, specifically prednisone and prednisolone. Thus cortisol-21-hemisuccinate was employed in the antibody purification processes:

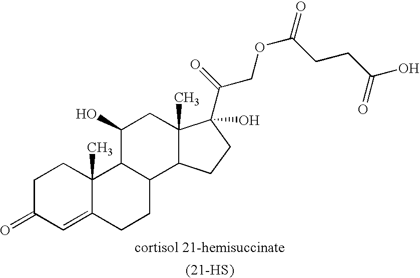

TABLE 4
Percent cross reactivity determined by 2000 ng/mL sample.

| | | 7 | 9 | 10 | 13 | 17 | Access |
|---|---|---|---|---|---|---|---|
| Syn-1 | 6a-methylprednisone | 9 | 2 | high | 10 | 3 | 2 |
| Syn-2 | prednisone | 1 | 21 | 0 | 1 | 20 | 3 |
| Syn-3 | triamcinolone acetonide | 0 | 1 | 0 | 1 | 0 | 0 |
| Syn-4 | triamcinolone | 0 | 1 | 0 | 0 | 0 | 0 |
| Syn-5 | fludrocortisone | 68 | 3 | 3 | 67 | 3 | 1 |
| Syn-6 | cortisone | 1 | 13 | 0 | 1 | 11 | 5 |
| Syn-7 | prednisolone | 5 | 15 | 68 | 4 | 14 | 21 |
| Syn-8 | fluoromethalone | 0 | 0 | 6 | 0 | 0 | 0 |
| Syn-9 | betamethasone | 0 | 0 | 0 | 0 | 0 | 0 |
| Syn-10 | dexamethasone | 6 | 0 | 0 | 6 | 0 | 0 |

TABLE 5
Percent cross reactivity determined from slopes of
([apparent cortisol] vs [cross reactant]).

| | | % cross reactivity | | | | | | |
|---|---|---|---|---|---|---|---|---|
| | | 7 | 9 | 10 | 13 | 17 | 21509 | ECB |
| SYN-1 | 6-AMP | 7 | 2 | 109 | 6 | 1 | — | — |
| SYN-2 | prednisone | <1 | 26 | 1 | — | — | <1 | 1 |
| SYN-5 | fluodrocortisone | | | | — | — | | |
| SYN-6 | cortisone | 1 | 15 | <1 | 1 | 13 | — | — |
| SYN-7 | prednisolone | 5 | 37 | 133 | 5 | 35 | — | — |
| SYN-10 | dexamethasone | | | | — | — | | |

Once the cross reactivities were determined for each of the antibodies, mathematical algorithms were provided, as described in the above Algorithm section, for their use in a multiplexed assay to determine true cortisol concentrations, by reducing the effect of the cross reacting species. Values are displayed below in Table 6:

TABLE 6
Cross reactivity algorithm.
Model for predicting Cortisol with Prednisolone
y = β₁x₁ + β₂x₂ + β₃x₃ + ε

| | Full Model | | Reduced Model | |
|---|---|---|---|---|
| Parameter | Value | P-Value | Value | P-Value |
| Antibody 7 | 1.1257 | 0 | 1.1248 | 0 |
| (Beta1) | | | | |
| Antibody 9 | −.1602 | 6.8044*10⁻²⁷. | −.1590 | 6.8044*10⁻²⁷ |
| (Beta2) | | | | |
| Antibody 10 | .0002538 | .9399 | — | — |
| (Beta3) | | | | |

B1 => Antibody 7
B2 => Antibody 9
B3 => Antibody 10

Specific antibodies were then selected to form the algorithm which agreed with the model as can be seen in Table 7 for the matrix accordingly.

TABLE 7

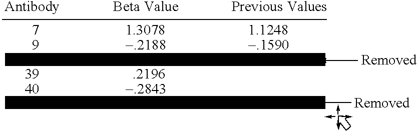

y = β₁χ₁ + β₂χ₂ + β₃χ₃ + β₄χ₄ + β₅χ₅ + β₆χ₆ + ε

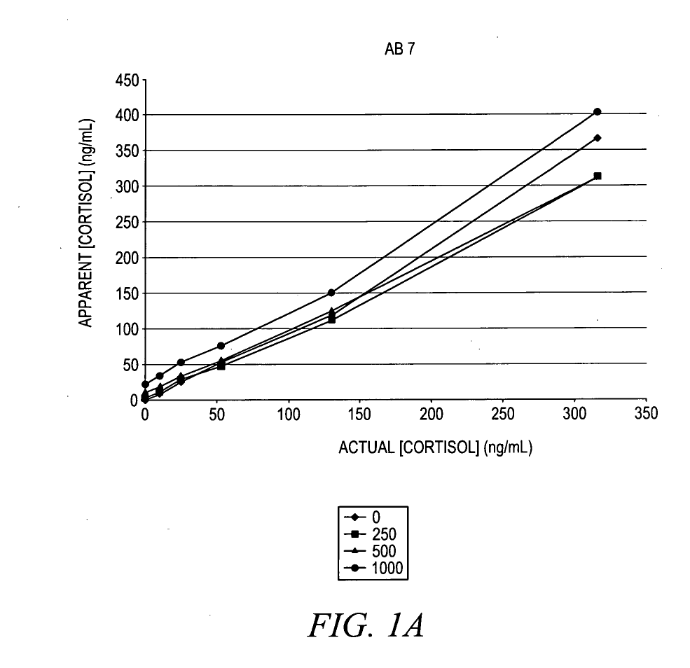

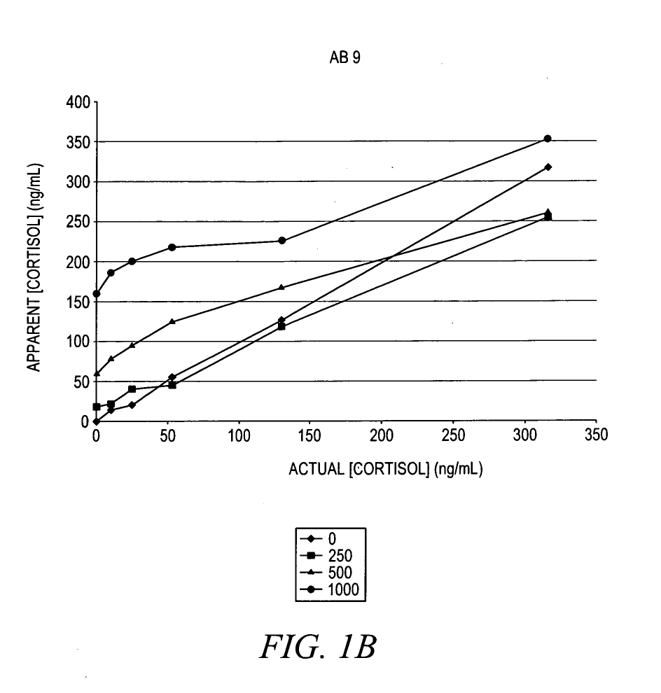

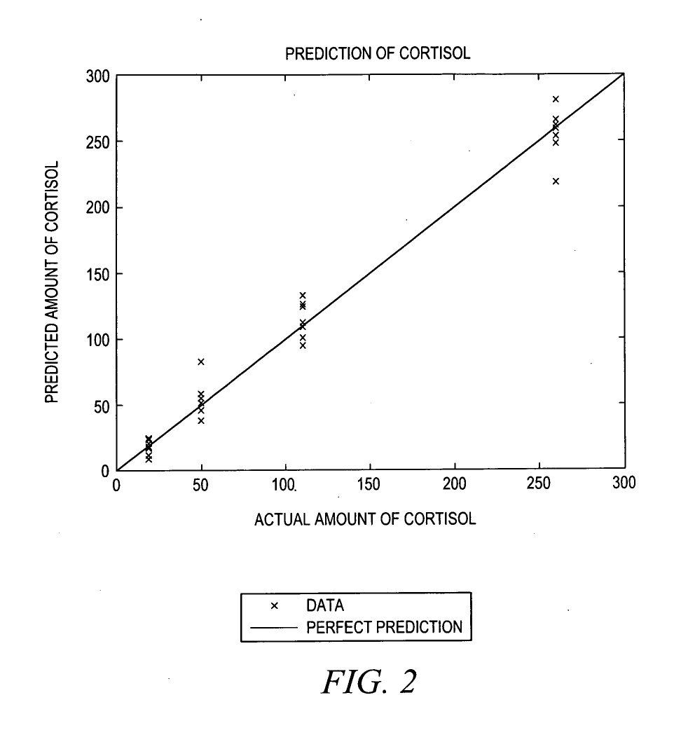

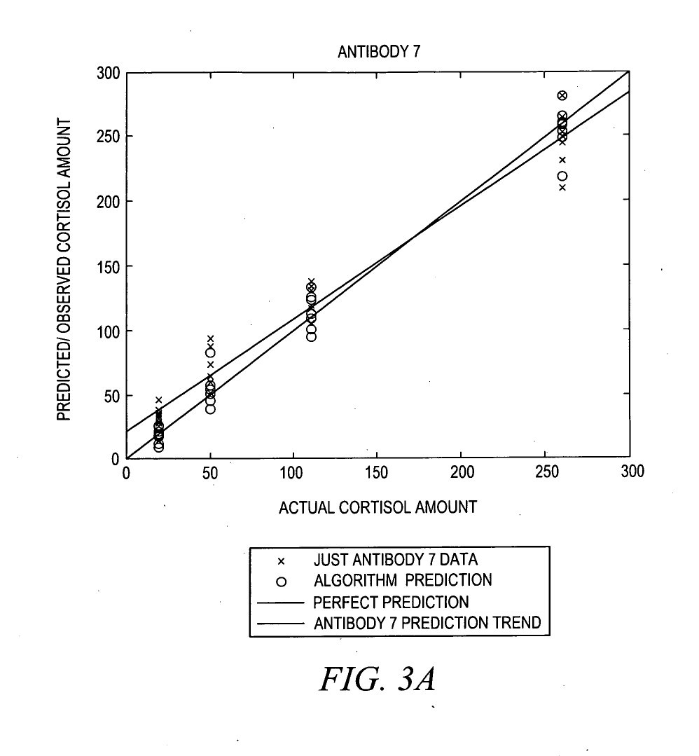

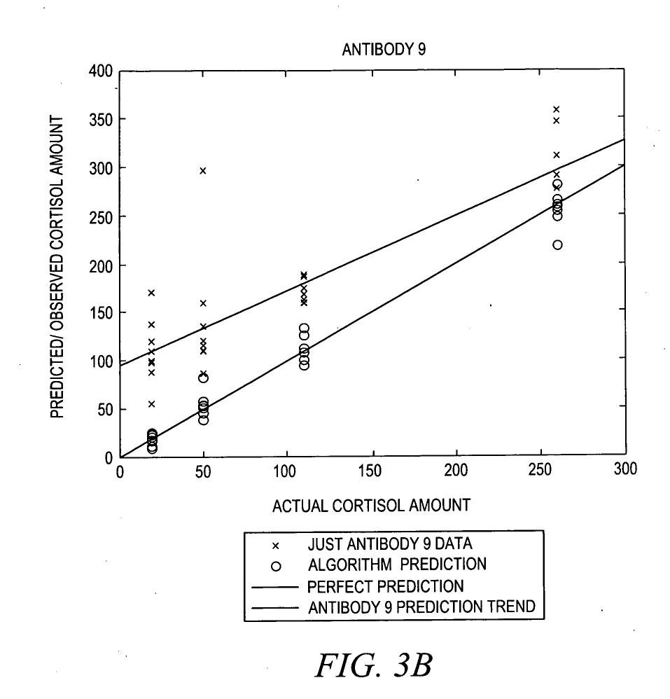

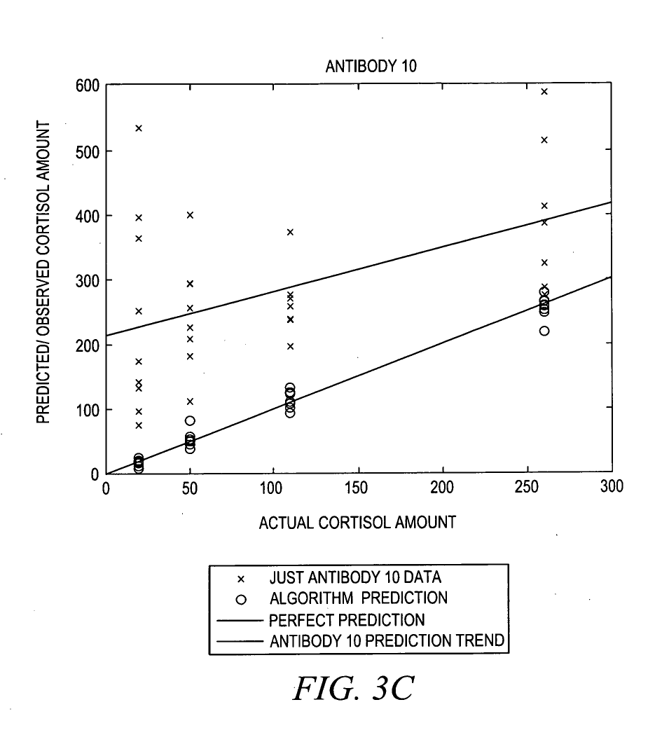

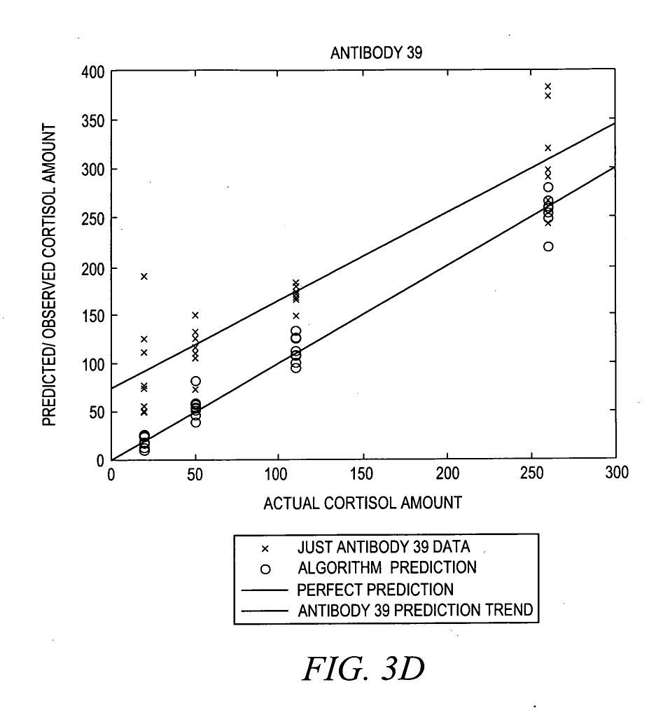

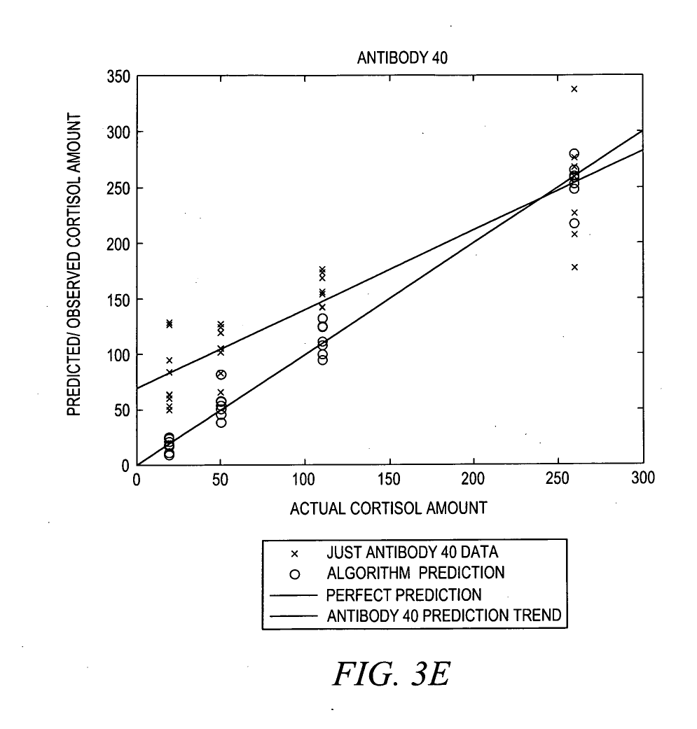

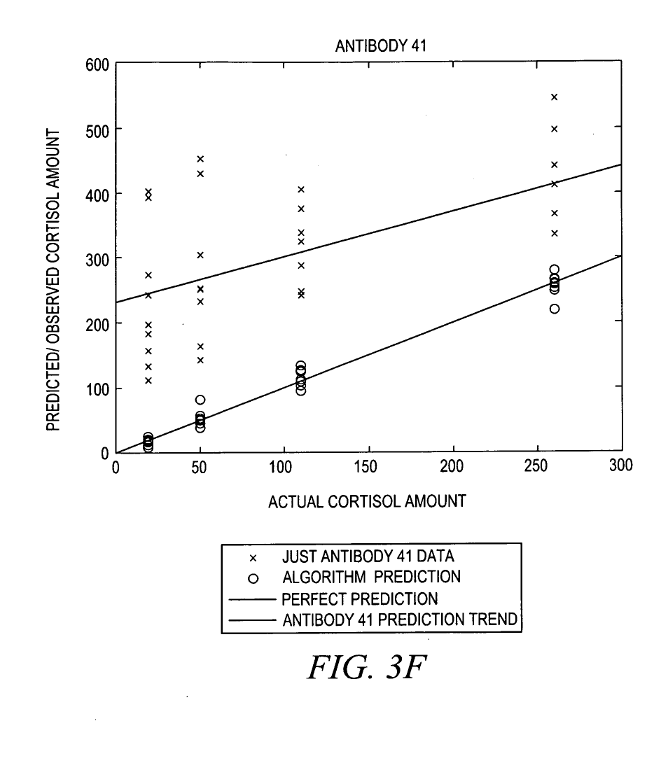

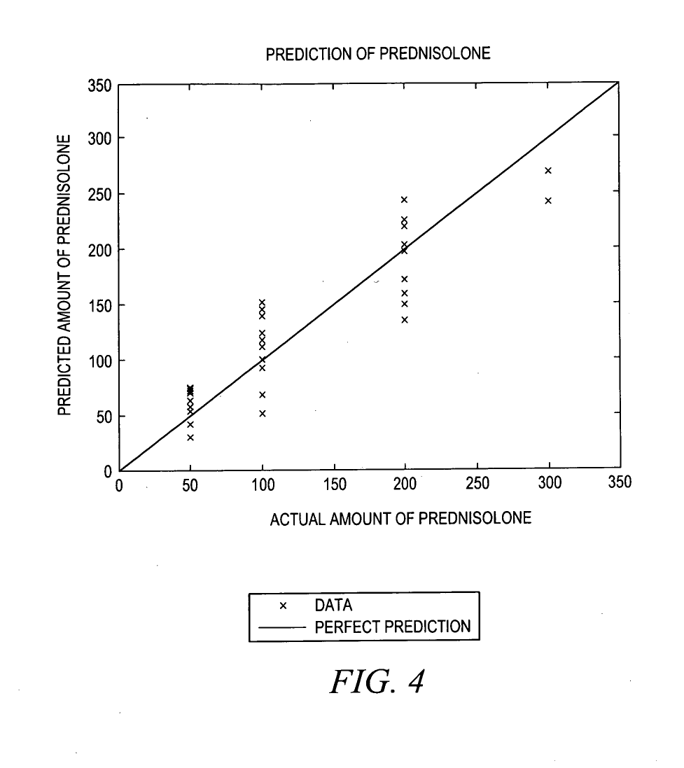

The results from these experiments are shown in FIGS. 1-5. The observations from the examples/figures illustrate the utility for an immunodiagnostic capable of performing operations to back calculate true concentrations for various analytes. FIGS. 3 (A-F) depict the true cortisol amount versus the single antibody estimated concentration (denoted with an x for each sample), the antibody prediction line (which does not intersect the Y-axis at 0) is the overall trend between estimated and actual concentration of cortisol for all samples and the perfect prediction line (which does intersect the Y-axis at 0) is the line that represents a trend of perfect prediction of cortisol. The circles show the estimated cortisol concentration versus actual concentration with the reduced regression model.

It is apparent from these results that use of the competitive immunoassay described herein, greatly improves the accuracy by which analyte levels, such as cortisol, are determined. Notably, as shown in FIGs. 3 (A-E), the actual cortisol levels and those determined by the methods of the present invention are very close, if not identical, whereas cortisol levels determined by existing commercial assays are often significantly off, sometimes estimating cortisol levels to be 100-5,000 fold greater than what is actually present. Subsequent clinical decisions based on literal interpretation of results obtained with existing commercial assays could result in dire consequences for the patients. Accordingly, the present invention has significant implications in improving diagnostic methods associated with detection of analytes in the presence of competitive analogs, which is expected to vastly improve clinical outcomes for affected patients.

Each of the aforementioned references are hereby incorporated by reference as if set forth fully herein.

1. A method for determining the concentration of an analyte in a test sample comprising the analyte and a plurality of competitive ligands, the method comprising:
contacting the test sample with at least two different anti-analyte antibodies, wherein each of the antibodies bind the analyte and have a different level of cross-reactivity for the competitive ligands;
detecting binding of the analytes and competitive ligands to the antibodies, thereby determining an observed analyte binding amount for each antibody; and
performing a regression analysis on the observed analyte binding amount for each antibody to determine the concentration of the analyte in the test sample.

2. The method of claim 1, wherein the analyte is cortisol and the competitive ligands are non-cortisol steroids.

3. The method of claim 1, wherein the regression analysis is linear regression.

4. The method of claim 1, wherein the regression analysis is non-linear regression.

5. The method of claim 1, wherein the regression analysis is displayed graphically.

6. The method of claim 1, wherein the regression analysis comprises solving the formula:
Y=Σβₙxₙ+c
wherein,
Y is the cortisol concentration;
n is the number of antibodies;
x is the observed steroid amount for each antibody;
β is the level of cross-reactivity for each antibody;
c is a calibration constant; and
Σ is the sum of βx for all antibodies.

7. The method of claim 1, wherein the test sample is plasma, serum, saliva, or urine.

8. The method of claim 2, wherein the non-cortisol steroids are selected from the group consisting of prednisolone, cortisone, 6-a methylprednisolone (6-AMP), progesterone, prednisone, fludrocortisone and dexamethasone.

9. The method of claim 2, wherein the test sample is contacted with cortisol comprising a label or prednisolone comprising a label prior to the detecting step.

10. The method of claim 9, wherein the label is a fluorescent label.

11. The method of claim 9, wherein the label is an enzyme.

12. The method of claim 9, wherein the label is alkaline phosphotase or horseradish peroxidase (HRP).

13. The method of claim 9, wherein the label comprises a xanthene, an indole, a benzofuran, a cyanine, a coumarin, a borapolyazaindacene, a phycobilliprotein, or a semiconductor nanocrystal.

14. The method of claim 9, wherein the label is bound to cortisol or prednisolone through a carboxymethyloxime linker.

15. The method of claim 9, wherein the label is bound to the cortisol or prednisolone through a succinate linker.

16. The method of claim 9, wherein the cortisol comprising a label is: 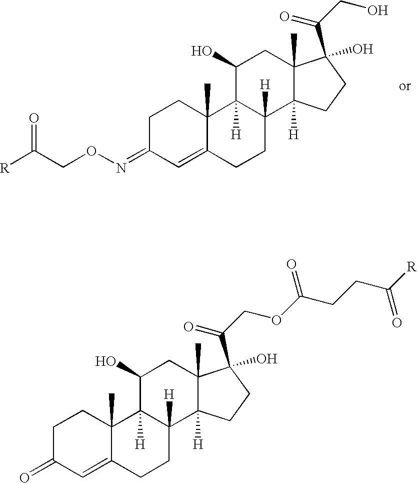
wherein,
R is a label.

17. The method of claim 9, wherein the prednisolone comprising a label is: 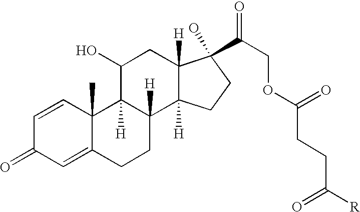
wherein,
R is a label.

18. The method of claim 9, wherein the label emits a detectable wavelength which corresponds to a signal intensity.

19. The method of claim 18, wherein the observed steroid binding amount is inversely proportional to the signal intensity.

20. The method of claim 19, wherein the signal intensity from the test sample is compared with an intensity obtained from a control sample having a known concentration of steroids.

21. The method of claim 1, wherein the antibodies are monoclonal antibodies.

22. The method of claim 1, wherein the antibodies are polyconal antibodies.

23. The method of claim 1, wherein the antibodies are immobilized on a solid support.

24. The method of claim 23, wherein the solid support is comprised of acrylamide, agarose, cellulose, nitrocellulose, glass, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, or polyamino acids.

25. The method of claim 23, wherein the solid support is a bead.

26. The method of claim 23, wherein the solid support further comprises at least one of a thin film, membrane, bottles, dishes, fibers, woven fibers, shaped polymers, particles, beads, or microparticles.

27. The method of claim 2, wherein the contacting step is performed in a buffered solution.

28. The method of claim 1, wherein the regression analysis is performed by a computer.

29. The method of claim 2, wherein the anti-steroid antibodies are produced by immunization of a mammal with a succinate bound steroid.

30. The method of claim 1, wherein the test sample is contacted with at least three antibodies.

31. The method of claim 1, wherein the test sample is contacted with at least five antibodies.

32. The method of claim 2, wherein the test sample is from an individual suspected of having or diagnosed with Cushing's syndrome or Addison's disease.

33. The method of claim 2, wherein the test sample is from an individual receiving treatment to modulate cortisol levels.

34. The method of claim 33, wherein the treatment comprises administration of hydrocortisone, Prednisone or Relacore.

35. The method of claim 2, wherein the test sample is from an individual that has hypercortisolism or hyporcortisolism.

36. A composition comprising at least three different isolated anti-steroid antibodies, wherein each of the antibodies bind cortisol and have a different level of cross-reactivity with non-cortisol steroids.

37. The composition of claim 36, further comprising labeled cortisol or labeled prednisolone.

38. The composition of claim 37, further comprising a test sample from an individual.

39. The composition of claim 36, wherein the antibodies are present in admixture.

40. The composition of claim 36, wherein the antibodies are immobilized on a solid support.

41. An array device comprising a solid support comprising at least two different anti-steroid antibodies, wherein each of the antibodies bind cortisol and have a different level of cross-reactivity with non-cortisol steroids.

42. A kit for determining cortisol concentration in a test sample comprising:
a solid support comprising at least two different anti-steroid antibodies, wherein each of the antibodies bind cortisol and have a different level of cross-reactivity with non-cortisol steroids; and
instructions on how to determine the cortisol concentration in the test sample.

43. The kit of claim 42, further comprising labeled cortisol or labeled prednisolone.

44. The kit of claim 42, further comprising a buffer solution.

45. The kit of claim 42, further comprising a computer for performing calculations to determine the cortisol concentration in the test sample.

46. The kit of claim 42, further comprising a control sample.

47. A method for detecting cortisol levels in an individual, the method comprising:
contacting a test sample from the individual with at least two different anti-steroid antibodies, wherein each of the antibodies bind cortisol and have a different level of cross-reactivity with non-cortisol steroids;
detecting binding of steroids to each of the antibodies, thereby determining an observed steroid binding amount for each of the antibodies;
performing a regression analysis on the observed steroid binding amount for each antibody to determine the concentration of cortisol in the test sample; and
comparing the concentration of cortisol in the test sample from the individual with cortisol levels in a control sample to detect cotisol levels in the individual.

48. A method of using a computer processor to determine the concentration of cortisol in a test sample, the method comprising:
(a) receiving data representing observed steroid concentrations in a test sample, wherein the data is obtained from contacting at least two different anti-steroid antibodies with a test sample, wherein each of the antibodies bind cortisol and have a different level of cross-reactivity with non-cortisol steroids; and
(b) performing a linear regression analysis with the computer processor with the data to determine a result comprising the concentration of cortisol in the test sample.

49. The method of claim 48, wherein the regression analysis comprises solving the formula:
Y=Σβₙxₙ+c
wherein,
Y is the cortisol concentration;
n is the number of antibodies;
x is the observed steroid amount for each antibody;
β is the level of cross-reactivity for each antibody;
c is a calibration constant; and
Σ is the sum of βx for all antibodies.

50. The method of claim 49, wherein the result is displayed graphically.

51. A method for determining the concentration of cortisol in a test sample, the method comprising:
contacting the test sample with at least two different anti-steroid antibodies, wherein each of the antibodies bind cortisol and have a different level of cross-reactivity with non-cortisol steroids;
detecting binding of steroids to the antibodies, thereby determining an observed steroid binding amount for each antibody; and
performing a regression analysis on the observed steroid binding amounts for each antibody to determine the concentration of cortisol in the test sample.
